(19) United States
(12) Patent Application Publication
(10) Pub. No.: US 2005/0033154 A1
(43) Pub. Date: Feb. 10, 2005

(54) Methods for measurement of magnetic resonance signal perturbations

(76) Inventor: Richard Christopher deCharms, Montara, CA (US)

Correspondence Address: WILSON SONSINI GOODRICH & ROSATI, 650 PAGE MILL ROAD, PALO ALTO, CA, 943041050, US

(21) Appl. No.: 10/861,786

(22) Filed: Jun. 3, 2004

Related U.S. Application Data
(60) Provisional application No. 60/475,931, filed on Jun. 3, 2003.

(51) Int. Cl. A61B 5/05

(52) U.S. Cl. 600/410

(57) ABSTRACT

The present invention relates to methods, software and systems for monitoring fluctuations in magnetic resonance signals. These methods may be used for measurements of the human brain and nervous system, and may be used for measuring electric currents and electromagnetic fields internal to an object. This method may include the use of a reference signal to accomplish differential recording of electromagnetic fields from two or more spatial locations.

### CROSS-REFERENCE

This application claims the benefit of U.S. Provisional Application, entitled “Methods For Physiological Monitoring—EmfMRI,” filed May 15, 2004 and U.S. Provisional Application No. 60/475,931, filed Jun. 3, 2003.

This application is also related to the following co-pending patent applications: U.S. Ser. No. 10/628,875, filed Jul. 28, 2003, now U.S. Publication No. US-2004/0092809 A1, entitled “Methods for Measurement and Analysis of Brain Activity”, and U.S. Ser. No. 10/066,004, filed Jan. 30, 2002, now U.S. Publication No. US-2002/0103429 A1, entitled “Methods for Physiological Monitoring, Training, Exercise and Regulation”, each of which is incorporated herein by reference in its entirety.”

### SUMMARY OF THE INVENTION

The present invention is directed to various methods relating to the measurement of fluctuations of magnetic resonance signals. These fluctuations may be used to measure fluctuations induced by electrical current and electromagnetic fields, and may be used to measure electrophysiological activity in the brain or nervous system.

In some embodiments, the present invention relates to a device to measure neuronal currents. Such device can include, for example, a means for reference MR signal amplification, a means for test MR signal amplification, and a means for determining the difference between the reference MR signal and the test MR signal. In various embodiments the reference MR signal and the test MR signal may be measured simultaneously. In some embodiments, the neuronal currents are induced by a neural activation (e.g., a neuronal activation can be selected from the group consisting of a visual image, a visual sequence, an auditory sound, an auditory sequence, a tactile sensation, an electrical stimulus to a peripheral location, an electrical stimulus to the central or peripheral nervous system, a pharmacological or other physiological stimulus, a perceptual stimuli, an instruction, and a set of instructions). In some embodiments, the device includes means for determining free induction decay of the amplified reference MR signal and amplified test MR signal. In some embodiments, the device includes means for determining free induction decay of the amplified reference MR signal and amplified test MR signal in substantially real time. In some embodiments, the device includes means for differentially measuring at least two MR signals.

In some embodiments, the present invention involves a device comprising means for measuring at least two MR signals and means for comparing at least two MR signals. Such a device can have means for measuring at least two MR signals simultaneously. Such a device can have means for measuring at least two MR signals after a stimulus. Examples of stimulus include, but are not limited to, visual image, a visual sequence, an auditory sound, an auditory sequence, a tactile sensation, an electrical stimulus to a peripheral location, an electrical stimulus to the central or peripheral nervous system, a pharmacological or other physiological stimulus, a perceptual stimuli, an instruction, and a set of instructions. The above device can further comprise means for amplifying at least two MR signals. Such a device can further comprise means for determining free induction decay of at least two MR signals in substantially real time. Such a device can further comprise an amplifier and a computing unit, wherein the computing unit compares at least two MR signals from at least two sources. The two or more MR signals can be from at least one voxel or at least two voxels. Such a device can have a computing unit that compares at least two MR signals by differentially measuring at least two MR signals following a single RF excitation. In some embodiments, the two or more MR signals are separated in time by 0.01, 0.1, 1, 5, 10, 100, 1000, or 10000 ms. Such a device can have a computing unit that differentially measures at least two MR signals in a substantially real time. Such a device can also have a computing unit that differentially measures at least two MR signals within a time period of less than 10 seconds.

In some embodiments, the present invention relates to a method for measuring a MR perturbation, wherein such method comprises the step of differentially measuring MR signals from at least two receivers from an object. Furthermore, in some embodiments, at least one receiver receives MR signals from a reference location and at least one receiver receives MR signal from a test location. In some embodiments, the above method further comprises the step of applying RF to the reference locations and the test locations. In some embodiments, the above RF produces free induction decay data from the reference locations and the test locations. In some embodiment, the above methods further comprise the step of converting the free induction decay to a series of phase or magnitude measurements per time period. In some embodiments, free induction decay data is analyzed in substantially real time or in less than 10 seconds. In some embodiments, the MR signals are measured immediately after a stimulus. In some embodiments, such stimulus is selected from the group consisting of a visual image, a visual sequence, an auditory sound, an auditory sequence, a tactile sensation, an electrical stimulus to a peripheral location, an electrical stimulus to the central or peripheral nervous system, a pharmacological or other physiological stimulus, a perceptual stimuli, an instruction, and a set of instructions. In some embodiments, the above methods further comprise the step of comparing MR signals prior to presentation of a stimulus to MR signals immediately following the presentation of the stimulus. The MR signals in any of the methods herein may be received simultaneously, amplified, or preferably, amplified before they are differentially measured. Any of the methods herein can be used to detect or localize MR signals in an object, such as a circuit, a living organism, tissue, or organ (e.g., brain or heart). When measuring at least two MR signals such signals are preferably separated in time by 0.01, 0.1, 1, 5, 10, 100, 1000, or 10000 ms. Measurements preferably occur in a substantially real time or in less than 10 seconds.

The present invention also relates to a method for diagnosing an individual susceptible or experiencing a central nervous system condition comprising the step of differentially measuring MR signals from the individual using at least two receivers. A central nervous system condition can be one that is selected from the group of conditions identified in FIG. 16. The above method can be accomplished using one or more receivers to receive an MR signal from a region of the brain selected from the group consisting of the regions identified in FIG. 15. The above method may further include the step of selecting a target voxel. Preferably the target voxel is selected using anatomical localizer images or functional localizer images. Furthermore, the above method may further include the step of comparing differential measurements of MR signals from the individual susceptible or experiencing a central nervous system condition and a healthy individual. The above method may further include the step of differential measuring, which occurs in real time. The above method contemplates real time measurements to be used to adjust an MR measurement parameter.

In some embodiments, the invention herein contemplates a method for localizing neuronal currents, wherein the method comprises the steps of: receiving an MR signal from a receiver; amplifying the MR signal; converting the MR signal into complex MR data; and comparing the data with an independent reference signal to obtain a differential measurement of MR signal. In some embodiments, the independent reference signal may be obtained by means other than MR imaging, such as from a gradiometer or a magnetometer. The MR signal and the independent reference signal are preferably made less than 100 seconds apart. The MR signal can further be used to produce a free induction decay. The above method and any other method herein may also include the step of providing a stimulus. Such may be time-synchronized following an RF excitation.

In some embodiments, the invention herein includes a method for measuring neuronal currents comprising the steps of: receiving at least two MR signals from at least one different voxels using at least one receiver during the same readout period; amplifying the MR signals; converting the MR signals into complex MR data; and comparing the complex MR data. Such methods may further include the step of producing a free induction decay for each MR signal. The receiving step can involve the use of at least two receivers. This and other methods herein can also include the step of comparing complex MR data with data collected from a physiological measurement selected from the group consisting of functional magnetic resonance imaging (fMRI), BOLD imaging, PET, SPECT, EEG (electroencephalogram) recordings or event-related electrical potentials, MEG recordings (magnetoencephalogram), electrode-based electrophysiological recording methods including single-unit, multi-unit, field potential or evoked potential recording, infrared or ultrasound based imaging methods. This and all other methods herein can also include the step of using real time measurements to adjust MR measurement parameters.

Any of the methods herein may be preformed by a programmable computer.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is an overview diagram of methods, components and processes of this invention.

FIG. 2 is an overview of the theory of the phase and magnitude response to an electric current.

FIG. 3 is a flow-chart of the process of setup and data acquisition.

FIG. 4 is a flow-chart of the process of data analysis.

FIG. 5 depicts the use of data from a reference location to correct data from one or more source location(s).

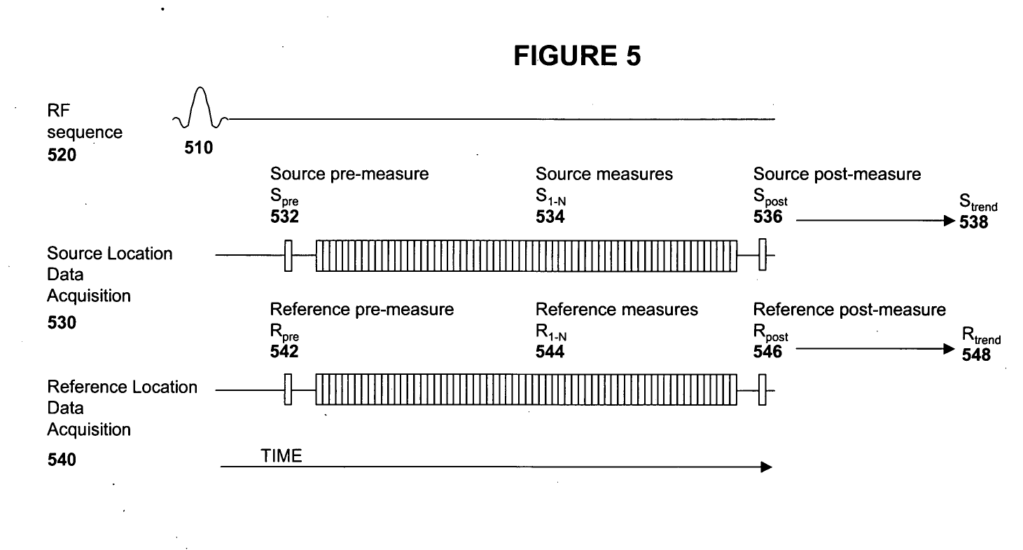

FIG. 6 depicts the flow of measurement data for the computation of MR perturbations.

FIG. 7 depicts example in vitro MR phase timecourse data.

FIG. 8 depicts example data of the correlation in phase noise between two receivers.

FIG. 9 depicts example MR phase timecourse data with and without differential recording.

FIG. 10 depicts example MR phase timecourse data from the visual cortex with and without the presentation of a visual stimulus.

FIG. 11 depicts the graphical prescription of target and reference voxels for differential MR measurements.

FIG. 12 depicts example stimulation and data acquisition protocols.

FIG. 13 depicts the difference between single-ended, differential, and differential filtered measurement using electrophysiology and MR physiology.

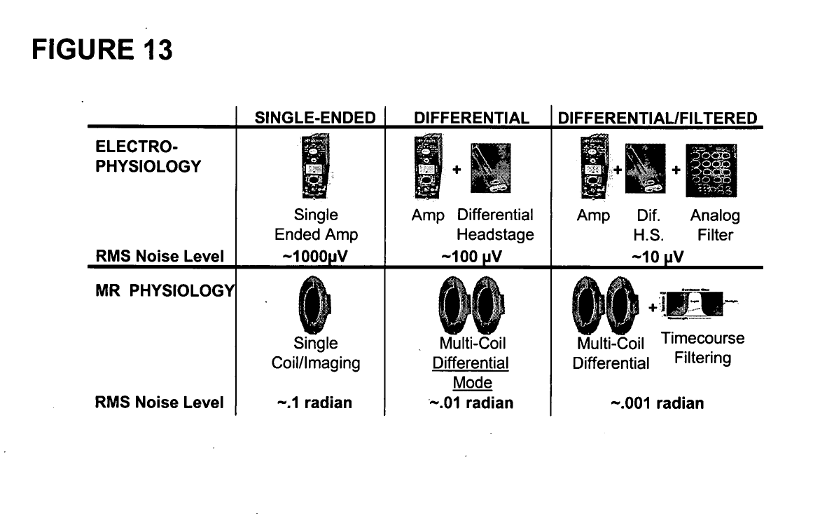

FIG. 14 depicts a conceptual overview of systems and methods of this invention.

FIG. 15 depicts a list of brain regions associated with central nervous system conditions.

FIG. 16 depicts examples of central nervous system conditions.

### DETAILED DESCRIPTION OF THE INVENTION

Definitions

Activity, as used herein, refers to physiological activity associated with one or more voxels of the brain whose physiological activity may be monitored. Examples of types of physiological activity include, but are not limited to, neuronal activity, blood flow, blood oxygenation, electrical activity, chemical activity, tissue perfusion, the level of a nutrient or trophic factor, the production or distribution of a trophic factor, the production, release, or reuptake of a neurotransmitter or neuromodulator, the growth of tissue such as neurons or parts of neurons, neural plasticity, and other physiological processes. Other examples are provided herein.

Activation, as used herein, refers to a change in activity in one or more voxels of the brain whose physiological activity may be monitored. This change may include an increase or decrease. It is noted that this change may also include a change where some voxels increase in activation at the same time that other voxels decrease in activation.

Activity metric, as used herein, refers to any computed measure of activity of one or more regions of interest of the brain.

Behavior, as used herein, refers to a physical or mental task or exercise engaged in by a subject, which may be in order to activate one or more regions of interest of the brain. Examples of different types of behaviors include, but are not limited to sensory perception, detection or discrimination, motor activities, cognitive processes such as mental imagery or mental manipulation of an imagined object, reading, emotional tasks such as attempting to create a particular affect or mood, verbal tasks such as listening to, comprehending, or producing speech. Other examples of behaviors are provided herein.

BOLD, as used herein refers to Blood Oxygen Level Dependent signal. This signal is typically measured using a functional magnetic resonance imaging device.

CSI, as used herein, refers to chemical shift imaging. This method may be used to measure MR spectra, or the time course of MR data, from more than one location in an object substantially simultaneously. This may be accomplished using phase encoding of spatial location, for example as implemented with PRESS-CSI.

Differential signal measurement, as used herein, refers to the comparison of measurements from one or more reference location or receiver with the measurements from one or more source location or receiver to determine differences between them.

FID, as used herein, refers to a free induction decay MR signal.

Instructions, as used herein, refers to any instruction to perform a physical or mental action that is communicated to a subject or an operator assisting a subject. Examples of instructions include, but are not limited to instructions to a subject to perform a behavior; instructions to a subject to rest; instructions to a subject to move; instructions to a subject to make a computer input; instructions to a subject to activate a brain region, such as to a designated level. Further examples of instructions are provided herein.

Localized region, as used herein refers to any region of the brain with a defined spatial extent. In one variation, a localized region measured by this invention may be internal relative to a surface of the brain.

MR, as used herein refers to magnetic resonance.

Pulse Sequence, as used herein refers to a sequence used to measure MR signals. A pulse sequence may include a sequence of RF pulses, and a sequence of x,y,z magnetic gradients, and a readout period during which MR data are collected.

Receiver, coil, receive coil, as used herein, refer to an antenna or means for collecting or measuring RF energy emanating from an object, such as might be used to measure MR signals. A receive coil may also transmit RF energy into the object, in the case of a transmit/receive coil.

Reference location, as used herein, refers to a location from where measurements are made within a subject that may be compared with measurements made at a source location. A reference location may be a location where a given perturbation of interest, for example an electromagnetic field, does not take place. This allows for differential measurement by making a comparison, such as a subtraction, from a source location. A reference location may be defined with respect to a source location either by using magnetic resonance imaging to define separate spatially defined voxels or regions of interest, or it may be defined through its physical spatial relationship to a receive element.

Region of interest or ROI or volume of interest, as used herein, refers to a particular one or more voxels of the brain of a subject. An ROI may occasionally be referred to as an area or volume of interest since the region of interest may be two dimensional (area) or three dimensional (volume). Frequently, it is an object of the methods of the present invention to monitor, control and/or alter brain activity in the region of interest. For example, the one or regions of interest of the brain associated with a given condition may be identified as the region of interest for that condition. In one variation, the regions of interest targeted by this invention are internal relative to a surface of the brain.

RF, as used herein, refers to radiofrequency energy, such as one or more pulses of radiofrequency energy produced by an MR scanner as part of MR measurement.

Scan volume, as used herein, refers to a three dimensional volume within which brain activity is measured. This volume may be divided into an array of voxels. For example, in the case of fMRI, a scanning volume may correspond to a 3-D cube (e.g., 22×22×12 cm) that comprises the volume of the head of a subject. This volume may be divided into a 64×64×17 array of subvolumes (voxels).

Source location, as used herein, refers to a location from where measurements are made within a subject. A source location may be a location where a given perturbation of interest, for example an electromagnetic field, is measured.

Single point, or location, as used herein, refers to an individual geometric locus or small area of volume, such as a single small geometric volume from which a physiological measurement may be made, with the volume being 0.1, 0.5, 1, 2, 3, 4, 5, 10, 15, 20, 30, 50, 100 mm in diameter. A device making a measurement from a single point is contrasted with a device making scanned measurements from an entire volume comprised of many single points.

Spatial array, as used herein, refers to a contiguous or non-contiguous set of location points, areas or volumes in space. The spatial array may be two dimensional in which case elements of the array are areas or three dimensional in which case elements of the array are volumes.

Stimulus information, as used herein, refers to any information which when communicated to a subject may cause the subject to have a perception, and/or to alter activity in one or more regions of interest of the subject's brain. Examples of stimulus information include but are not limited to: displays of static or moving images, sounds, and tactile sensations. It should be recognized that certain types of information may perform a dual function of being stimulus information and also communicating another type of information. A stimulus can also correspond to a physical stimulus, such as an electrical stimulus applied peripherally, or applied directly to peripheral or central neural tissue, or applied using magnetic means including transcutaneous magnetic stimulation. A stimulus can also correspond to a pharmacologic stimulus, such as the application of a drug or substance either locally, or systemically, or through the use of a controlled delivery device.

Stimulus set or behavior set, as used herein, refers to a defined set of stimuli or behaviors that are to be used to activate one or more particular regions of interest of a subject's brain. The exemplars forming the set may constitute either a set of discrete exemplars (such as a set of digitized photographic images of faces, instructions, or words), or a continuum from which particular exemplars can be drawn (such as the sound frequencies from 2000-8000 Hz or visual gratings with spatial frequency from 0.01-10 cycles/degree of arc). As will be described herein, a set of exemplars may be used to identify a subset that are found to more effectively activate the particular one or more particular regions of interest. A stimulus can also correspond to a physical stimulus, such as an electrical stimulus applied peripherally, or applied directly to peripheral or central neural tissue, or applied using magnetic means including transcutaneous magnetic stimulation. A stimulus can also correspond to a pharmacologic stimulus, such as the application of a drug or substance either locally, or systemically, or through the use of a controlled delivery device.

Subject, as used herein, refers to a person, animal, or physical object, whose MR signal is measured in conjunction with performing the methods of the present invention.

Substantially real time, as used herein, refers to a short period of time between process steps. Preferably, something occurs in substantially real time if it occurs within a time period of less than 10 seconds, more preferably less than 5, 4, 2, 1, 0.5, 0.2, 0.1, 0.01 seconds or less. In one particular embodiment, computing an activity metric is performed in substantially real time relative to when the brain activity measurement used to compute the activity metric was taken. In another particular embodiment, communicating information based on measured activity is performed in substantially real time relative to when the brain activity measurement was taken. Because activity metrics and information communication may be performed in substantially real time relative to when brain activity measurements are taken, it is thus possible for these actions to be taken while the subject is still in position to have his or her brain activity measured.

Trial, as used herein, refers to a single measurement sequence. For example, for a single-shot pulse sequence, a trial corresponds to a single application of RF energy to a sample and subsequent data readout. Multiple trials may be collected as part of measurement, and then averaged, possibly after processing, to produce better estimates of a value being measured.

Task or Behavior, as used herein, refers to a perceptual, cognitive, behavioral, emotional, or other activity undertaken by a subject, typically repetitively as part of a trial.

Voxel, as used herein, refers to a point or three-dimensional volume from which one or more measurements are made. This volume need not be spatially continuous. A voxel may be a single measurement point, or may be part of a larger three dimensional grid array that covers a volume. It should be noted that this is a specialized use of the term voxel, in that a measurement voxel may be a spatially defined volume that can have one, two or more spatially separated regions.

Description of Related Art

A variety of different brain scanning methodologies have been developed that may be used to identify changes of mental states or conditions including Positron Emission Tomography (PET) and Single Photon Emission Computed Tomography (SPECT), electroencephalogram (EEG) based imaging, magnetoencephalogram (MEG) based imaging, and functional magnetic resonance imaging (fMRI).

Potential Importance and Applications

A technology allowing for direct measurement of neuronal currents within the brain would represent a major technological breakthrough for functional neuroimaging, an area that has already led to a revolution in progress in cognitive neuroscience and related disciplines [Posner, Petersen et al. (1988). “Localization of cognitive operations in the human brain.” Science 240: 1627-31; Posner and Raichle (1998). “The neuroimaging of human brain function.” Proc Natl Acad Sci USA 95 (3): 763-4; Raichle (2001). “Functional Neuroimaging: A Historical and Physiological Perspective.” Handbook of functional neuroimaging of cognition.]. To date, there is no non-invasive technology for spatially resolved, high temporal resolution, direct measurement of neuronal signals from within the brain.

Neuronal signaling takes place on a characteristic timescale of several milliseconds to several hundred milliseconds [deCharms and Zador (2000). “Neural representation and the, cortical code.” Annu Rev Neurosci 23: 613-47.], and a central question in modem brain research is the role of the temporal characteristics of neuronal signals. This technology enables a wide variety of novel measures. Applications may include: 1) measurement of the timing and sequencing of neuronal activation across brain regions, 2) comparison of neuronal function with the BOLD FMRI response, 3) measurement of neuronal activation in white matter areas (where hemodynamics-based functional signals are limited), 4) direct measurement and localization of dipoles previously modeled using MEG/EEG data, 5) measurement and localization of fast evoked-responses in sub-cortical brain regions previously out of reach of localization using MEG/EEG, 6) measurement of neuronal correlation between different brain regions, 7) measurement and localization of EEG signals and generators within the brain during cognitive tasks (e.g. alpha band, gamma band), and, if SNR ultimately proves sufficient, 8) methods for precise spatial localization of neuronal activation not limited by hemodynamics.

The direct measurement of neuronal current may also have significant long-term applications in disease diagnosis. Some applications as a disease diagnostic may include: 1) localization of areas of functional impairment due to tumors, 2) localization of seizure foci, 3) mapping of the level of neurophysiological activity in peri-lesional areas surrounding cerebral infarct, tumor, or other lesion, 4) precise, non-invasive assessment of eloquent cortex during pre-surgical planning, e.g. preceding tumor or seizure focus resection, 5) monitoring of the therapeutic effect of treatment regimens that affect neural function, 6) pharmacological testing.

Brain Scanning Technologies

A variety of different brain scanning methodologies have been developed that may be used to identify changes of mental states or conditions including Positron Emission Tomography (PET) and Single Photon Emission Computed Tomography (SPECT), electroencephalogram (EEG) based imaging, magnetoencephalogram (MEG) based imaging, and functional magnetic resonance imaging (fMRI).

For example, magnetic resonance imaging (MRI) has been used successfully to study blood flow in vivo. U.S. Pat. Nos. 4,983,917, 4,993,414, 5,195,524, 5,243,283, 5,281,916, and 5,227,725 provide examples of the techniques that have been employed. These patents are generally related to measuring blood flow with or without the use of a contrast bolus, some of these techniques referred to in the art as MRI angiography. Many such techniques are directed to measuring the signal from moving moieties (e.g., the signal from arterial blood water) in the vascular compartment, not from stationary tissue. Thus, images are based directly on water flowing in the arteries, for example. U.S. Pat. No. 5,184,074, describes a method for the presentation of MRI images to the physician during a scan, or to the subject undergoing MRI scanning.

In the brain, several researchers have studied perfusion by dynamic MR imaging using an intravenous bolus administration of a contrast agent in both humans and animal models (See, A. Villringer et al, Magn. Reson, Med., Vol. 6 (1988), pp 164-174; B. R. Rosen et al, Magn. Reson. Med., Vol. 14 (1999), pp. 249-265; J. W. Belliveau et al, Science, Vol. 254 (1990), page 716). These methods are based on the susceptibility induced signal losses upon the passage of the contrast agent through the microvasculature. Although these methods do not measure perfusion (or cerebral blood flow, CBF) in classical units, they allow for evaluation of the related variable rCBV (relative cerebral blood volume). For example, in U.S. Pat. No. 5,190,744 to Rocklage, quantitative detection of blood flow abnormalities is based on the rate, degree, duration, and magnitude of signal intensity loss which takes place for a region following MR contrast agent administration as measured in a rapid sequence of magnetic resonance images.

With the advent of these brain scanning methodologies, blood flow in various brain areas has been effectively correlated with various brain disorders such as Attention Deficit Disorder (ADD), Schizophrenia, Parkinson's Disease, Dementia, Alzheimers Disease, Endogenous Depression, Oppositional Defiant Disorder, Bipolar Disorder, memory loss, brain trauma, Epilepsy and others.

The prior art also describes a variety of inventions dating back to the 1960's have provided a way allowing subjects to learn to control muscle, autonomic or neural activity through processes. Examples and descriptions are included in U.S. Pat. No. 4,919,143. U.S. Pat. No. 4,919,143, U.S. Pat. No. 5,406,957, U.S. Pat. No. 5,899,867 and U.S. Pat. No. 6,097,981.

Considerable research has also been directed to biological feedback of brainwave signals known as electroencephalogram (EEG) signals. One conventional neurophysiological study established a functional relationship between behavior and bandwidths in the 12-15 Hz range relating to sensorimotor cortex rhythm EEG activity (SMR). Sterman, M. B., Lopresti, R. W., & Fairchild, M. D. (1969). Electroencephalographic and behavioral studies of monomethylhdrazine toxicity in the cat. Technical Report AMRL-TR-69 3, Wright-Patterson Air Force Base, Ohio, Air Systems Command. A cat's ability to maintain muscular calm, explosively execute precise, complex and coordinated sequences of movements and return to a state of calm was studied by monitoring a 14 cycle brainwave. The brainwave was determined to be directly responsible for the suppression of muscular tension and spasm. It was also demonstrated that the cats could be trained to increase the strength of specific brainwave patterns associated with suppression of muscular tension and spasm. Thereafter, when the cats were administered drugs which would induce spasms, the cats that were trained to strengthen their brainwaves were resistant to the drugs.

The 12-15 Hz SMR brainwave band has been used in EEG training for rectifying pathological brain underactivation. In particular the following disorders have been treated using this type of training: epilepsy (as exemplified in M. B. Sterman's, M. B. 1973 work on the “Neurophysiologic and Clinical Studies of Sensorimotor EEG Biofeedback Training: Some Effects on Epilepsy” L. Birk (Ed.), Biofeedback: Behavioral Medicine, New York: Grune and Stratton); Giles de la Tourette's syndrome and muscle tics (as exemplified in the inventor's 1986 work on “A Simple and a Complex Tic (Giles de la Tourette's Syndrome): Their response to EEG Sensorimotor Rhythm Biofeedback Training”, International Journal of Psychophysiology, 4, 91-97 (1986)); hyperactivity (described by M. N. Shouse, & J. F. Lubar's in the work entitled “Operant Conditioning of EEG Rhythms and Ritalin in the Treatment of Hyperkinesis”, Biofeedback and Self-Regulation, 4, 299-312 (1979); reading disorders (described by M. A. Tansey, & Bruner, R. L.'s in “EMG and EEG Biofeedback Training in the Treatment of a 10-year old Hyperactive Boy with a Developmental Reading Disorder”, Biofeedback and Self-Regulation, 8, 25-37 (1983)); learning disabilities related to the finding of consistent patterns for amplitudes of various brainwaves (described in Lubar, Bianchini, Calhoun, Lambert, Brody & Shabsin's work entitled “Spectral Analysis of EEG Differences Between Children with and without Learning Disabilities”, Journal of Learning Disabilities, 18, 403-408 (1985)) and; learning disabilities (described by M. A. Tansey in “Brainwave signatures-An Index Reflective of the Brain's Functional Neuroanatomy: Further Findings on the Effect of EEG Sensorimotor Rhythm Biofeedback Training on the Neurologic Precursors of Learning Disabilities”, International Journal of Psychophysiology, 3, 85-89 (1985)). In sum, a wide variety of disorders, whose symptomology includes impaired voluntary control of one's own muscles and a lowered cerebral threshold of overload under stress, were found to be treatable by “exercising” the supplementary and sensorimotor areas of the brain using EEG biofeedback.

U.S. Pat. No. 5,995,857 describes an apparatus and method for providing biofeedback of human central nervous system activity using radiation detection. In this patent, radiation from the brain resulting either from an ingested or injected radioactive material or radio frequency excitation or light from an external source impinging on the brain is measured by suitable means and is made available to the subject on which the measurement is being made for his voluntary control. The measurement may be metabolic products of brain activity or some quality of the blood, such as its oxygen content. The system described therein utilizes red and infrared light to illuminate the brain through the translucent skull and scalp.

Spatial Imaging Techniques: PET and fMRI

PET imaging led to early excitement about the potential for non-invasive measurement of human brain activation [Posner, Petersen et al. (1988). “Localization of cognitive operations in the human brain.” Science 240: 1627-31; Posner and Raichle (1998). “The neuroimaging of human brain function.” Proc Natl Acad Sci USA 95 (3): 763-4; Raichle (2001). “Functional Neuroimaging: A Historical and Physiological Perspective.” Handbook of functional neuroimaging of cognition.], and has continued to be particularly important in allowing for measurement of physiological processes[Raichle (1987). “Circulatory and metabolic correlates of brain function in normal humans.” Handbook of Physiology: The Nervous System 5: 643-674; Jezzard and Song (1996). “Technical foundations and pitfalls of clinical fMRI.” Neuroimage 4 (3 Pt 3): S63-75; Raichle (1997). “Food for thought. The metabolic and circulatory requirements of cognition.” Ann N Y Acad Sci 835: 373-85; Posner and Raichle (1998). “The neuroimaging of human brain function.” Proc Natl Acad Sci USA 95 (3): 763-4; Raichle and Gusnard (2002). “Appraising the brain's energy budget.” Proc Natl Acad Sci USA 99 (16): 10237-9.]. In the 10 years since its inception, fMRI has become a dominant tool for brain mapping. In particular, the Blood Oxygenation Level Dependent (BOLD) method [Ogawa, Lee et al. (1990). “Brain magnetic resonance imaging with contrast dependent on blood oxygenation.” Proc Natl Acad Sci USA 87 (24): 9868-72; Belliveau, Cohen et al. (1991). “Functional studies of the human brain using high-speed magnetic resonance imaging.” J Neuroimaging 1 (1): 36-41; Kwong, Belliveau et al. (1992). “Dynamic magnetic resonance imaging of human brain activity during primary sensory stimulation.” Proc Natl Acad Sci USA 89 (12): 5675-9; Ogawa, Lee et al. (2000). “An approach to probe some neural systems interaction by functional MRI at neural time scale down to milliseconds.” Proc Natl Acad Sci USA 97 (20): 11026-31; Menon (2001). “Imaging function in the working brain with fMRI.” Curr Opin Neurobiol 11 (5): 630-6; Kim and Ogawa (2002). “Insights into new techniques for high resolution functional MRI.” Curr Opin Neurobiol 12 (5): 607-15.] has been adopted by a large number of institutions worldwide. FMRI is non-invasive, higher resolution than other methods[Menon and Goodyear (1999). “Submillimeter functional localization in human striate cortex using BOLD contrast at 4 Tesla: implications for the vascular point-spread function.” Magn Reson Med 41 (2): 230-5; Menon (2001). “Imaging function in the working brain with FMRI.” Curr Opin Neurobiol 11 (5): 630-6; Ugurbil, Toth et al. (2003). “How accurate is magnetic resonance imaging of brain function?” Trends Neurosci 26 (2): 108-14.], and requires no exogenous source of contrast fMRI and PET are inherently restricted by their physiological basis. Techniques based upon hemodynamics may be limited by the temporal characteristics of the brain hemodynamic response, which has a time constant of several seconds [Kim, Richter et al. (1997). “Limitations of temporal resolution in functional MRI.” Magn Reson Med 37 (4): 631-6.]. It is also not straightforward to determine the exact relationship between observed hemodynamic activations and underlying neural function [Boynton, Engel et al. (1996). “Linear systems analysis of functional magnetic resonance imaging in human V1.” J Neurosci 16 (13): 4207-21; Friston, Josephs et al. (1998). “Nonlinear event-related responses in FMRI.” Magn Reson Med 39 (1): 41-52; Vazquez and Noll (1998). “Nonlinear aspects of the BOLD response in functional MRI.” Neuroimage 7 (2): 108-18; Birn, Saad et al. (2001). “Spatial heterogeneity of the nonlinear dynamics in the FMRI BOLD response.” Neuroimage 14 (4): 817-26.]. Finally, reliance on hemodynamics may also create an inherent limit in spatial resolution governed by the vascular system.

MEG/EEG

MEG and EEG enable non-invasive measurement of neuronal currents with high temporal resolution, but more limited spatial capability. These techniques take measurements outside of the skull, so localization of current sources within the brain is based upon solutions to the non-unique inverse problem [Hamalainen, Hari et al. (1993). “Magnetoencephalography? Theory, instrumentation, and applications to noninvasive studies of the working human brain.” Rev. Mod. Phys. 65: 413-497; Stenbacka, Vanni et al. (2002). “Comparison of minimum current estimate and dipole modeling in the analysis of simulated activity in the human visual cortices.” Neuroimage 16 (4): 936-43.]. Spatial accuracy of MEG and EEG localization have been repeatedly estimated and compared, and are of order 3-20 mm for sources near the cortical surface [Leahy, Mosher et al. (1998). “A study of dipole localization accuracy for MEG and EEG using a human skull phantom.” Electroencephalogr Clin Neurophysiol 107 (2): 159-73; Liu, Belliveau et al. (1998). “Spatiotemporal imaging of human brain activity using functional MRI constrained magnetoencephalography data: Monte Carlo simulations.” Proc Natl Acad Sci USA 95 (15): 8945-50; Bonmassar, Schwartz et al. (2001). “Spatiotemporal brain imaging of visual-evoked activity using interleaved EEG and fMRI recordings.” Neuroimage 13 (6 Pt 1): 1035-43; Darvas, Schmitt et al. (2001). “Spatio-temporal current density reconstruction (stCDR) from EEG/MEG-data.” Brain Topogr 13 (3): 195-207; Fuchs, Wagner et al. (2001). “Boundary element method volume conductor models for EEG source reconstruction.” Clin Neurophysiol 112 (8): 1400-7; Gavit, Baillet et al. (2001). “A multiresolution framework to MEG/EEG source imaging.” IEEE Trans Biomed Eng 48 (10): 1080-7; Liu, Dale et al. (2002). “Monte Carlo simulation studies of EEG and MEG localization accuracy.” Hum Brain Mapp 16 (1): 47-62; Moradi, Liu et al. (2003). “Consistent and precise localization of brain activity in human primary visual cortex by MEG and fMRI.” Neuroimage 18 (3): 595-609.]. For deeper-lying structures, localization is considerably more problematic. A large literature has developed surrounding methods of source localization modeling [Williamson and Kautman (1981). “Biomagnetism.” J. Magn. Mat. 22: 129-201; Okada (1983). “Neurogenesis of evoked magnetic fields.” Biomagnetism: An Interdisciplinary Approach: 399-421; loannides (1993). “Brain function as revealed by current density analysis of magnetoencephalography signals.” Physiol Meas 14 Suppl 4A: A75-80; Onofij, Fulgente et al. (1995). “Visual evoked potentials generator model derived from different spatial frequency stimuli of visual field regions and magnetic resonance imaging coordinates of V1, V2, V3 areas in man.” Int J Neurosci 83 (3-4): 213-39; Uutela, Hamalainen et al. (1999). “Visualization of magnetoencephalographic data using minimum current estimates.” Neuroimage 10 (2): 173-80; Stenbacka, Vanni et al. (2002). “Comparison of minimum current estimate and dipole modeling in the analysis of simulated activity in the human visual cortices.” Neuroimage 16 (4): 936-43.], including a variety of techniques from the cruciform model [Okada (1983). “Neurogenesis of evoked magnetic fields.” Biomagnetism: An Interdisciplinary Approach: 399-421; Onoftj, Fulgente et al. (1995). “Visual evoked potentials generator model derived from different spatial frequency stimuli of visual field regions and magnetic resonance imaging coordinates of V1, V2, V3 areas in man;” Int J Neurosci 83 (3-4): 213-39.], to distributed source analysis and magnetic field tomography (MFT)[Moradi, Liu et al. (2003). “Consistent and precise localization of brain activity in human primary visual cortex by MEG and fMRI.” Neuroimage 18 (3): 595-609.], minimum norm estimates (MNE) that select the current distribution explaining the measured data with the smallest Euclidean norm of the currents [Hamalainen and Ilmoniemi (1994). “Interpreting magnetic fields of the brain: minimum norm estimates.” Med Biol Eng Comput 32 (1): 35-42.], and minimum current estimates (MCE) [Matsuura and Okabe (1995). “Selective minimum-norm solution to the biomagnetic inverse problem.” IEEE Trans Biomed Eng 42: 608-615; Stenbacka, Vanni et al. (2002). “Comparison of minimum current estimate and dipole modeling in the analysis of simulated activity in the human visual cortices.” Neuroimage 16 (4): 936-43.].

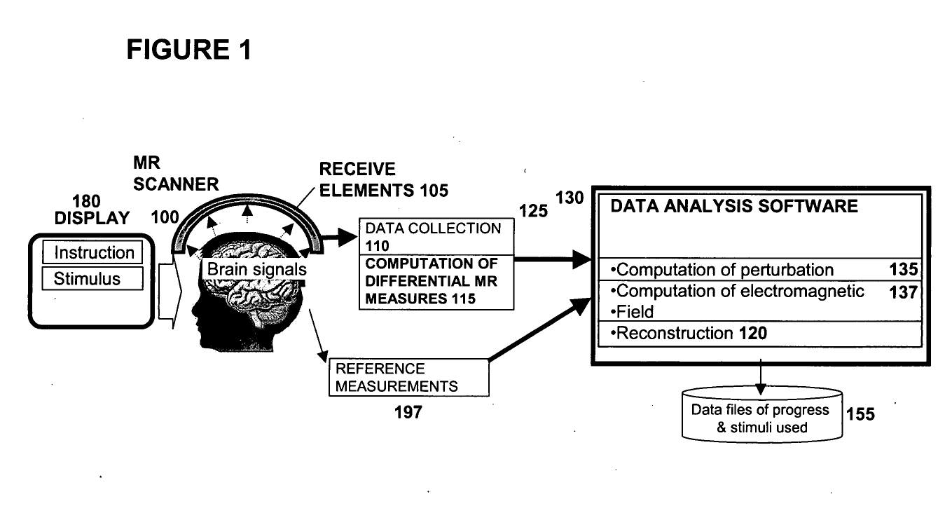

The brain is the seat of psychological, cognitive, emotional, sensory and motoric activities. Many psychological and neurological conditions arise because of inadequate levels of activity or inadequate control over discretely localized regions within the brain. The present invention provides methods, software, and systems that may be used to measure electrophysiological activity of one or more regions of interest. An overview diagram depicting the components and process of the invention is presented in FIG. 1. As illustrated, a scanner and associated control software 100 initiates scanning pulse sequences, makes resulting measurements from a plurality of receive elements 105 that may include amplification, and communicates resultant electronic signals associated with data collection software 110. Data from different receive elements, different spatial locations, and/or different time points may then be compared or subtracted to produce the result of differential MR measures 115. This data may then be converted to time series or image data corresponding to voxels, images or volumes of the brain by the reconstruction software 120. The resultant timeseries data, images or volume 125 may be passed to the data analysis software 130. The data analysis/behavioral control software may perform computations on the data to produce activity metrics that are measures of electrophysiological activity in brain regions of interest, electrical activity, or other MR perturbations. These computations include additional post-processing, including differential post-processing 135, computation of activation image/volumes 137, computation of activity metrics 140. The results and other information and ongoing collected data may be stored to data files 155. These measurements may take place as instructions or stimuli are presented to subjects. In addition, reference measurements at one or more reference location may also be made.

MR Perturbation Measurements

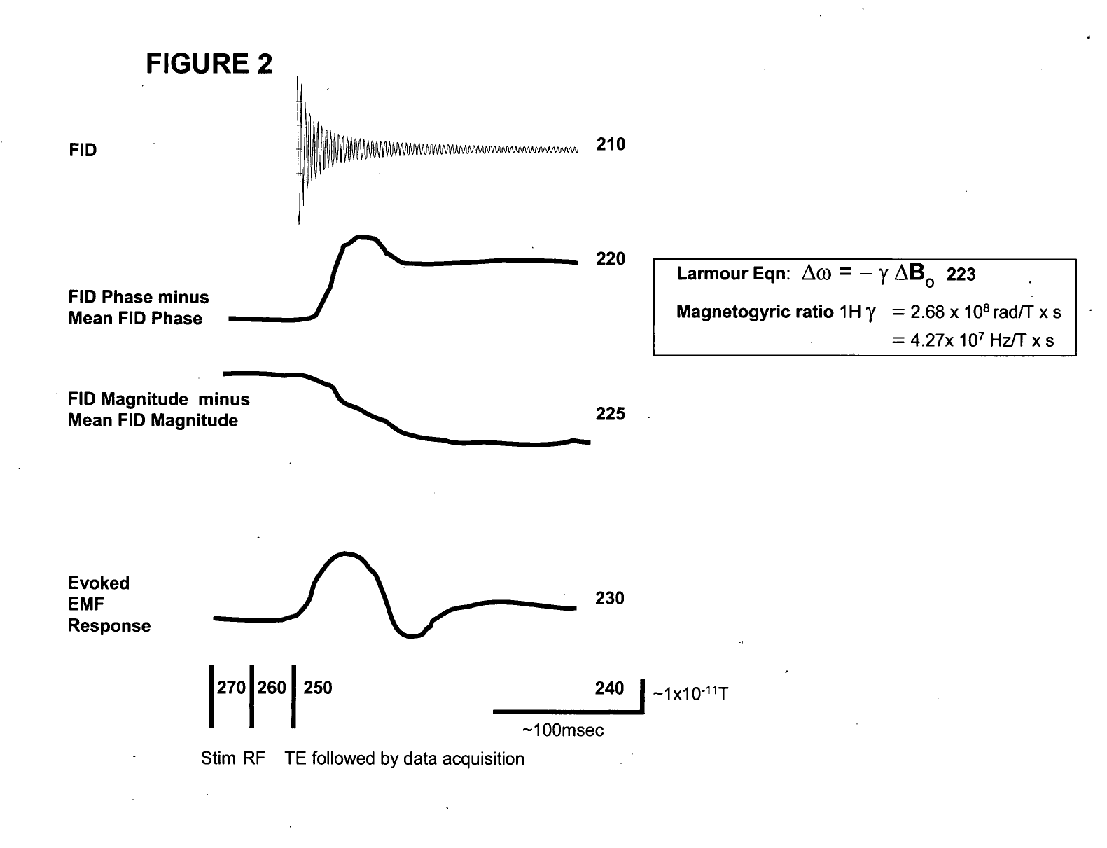

The basis of the measurements allowed by this invention may be the perturbation of magnetic resonance signals by the presence of an electromagnetic field. Example signals measured and temporal sequences involved are depicted in FIG. 2. An electromagnetic field may perturb a magnetic resonance signal, which may be measured as a free induction decay 210, in several ways.

The MR precession frequency of a substance being measured, for example hydrogen nuclei within the brain, may be altered by the magnetic field strength. Therefore, the resultant frequency of an MR signal may be very slightly changed by small electromagnetic fields because these fields change the local magnetic field experienced by the nuclei of the substance being measured. This frequency change may be read out as a change in the phase of the MR signal 220 with respect to some reference frequency, such as an estimate of the Larmour frequency of the substance being measured at the Bo field strength in the measurement instrument 223. A constant electromagnetic field perturbation may be measured as an ongoing increase or decrease in the phase of an MR signal relative to the reference Bo field strength in the absence of the electromagnetic field.

The magnitude of an MR signal measured from a substance 225 may also be changed by the presence of a changing electromagnetic field 230. This may take place because the electromagnetic field causing the change is not perfectly homogeneous within the volume from which the measurement is made (for example an imaging voxel or spectroscopy voxel). Since the electromagnetic field leads to a change in the homogeneity of the magnetic field, this can lead to susceptibility induced decreases in the signal intensity from the measured voxel. These may be measured using either gradient echo or spin echo methods or others.

The orientation of the MR signal may also be changed by the presence of an electromagnetic field. The vector representing the average orientation of nuclear precession of a substance may thereby be slightly changed by an electromagnetic field. Therefore, using two or more sensors that are sensitive to different spatial components of the orientation of the nuclear precession, the orientation of this vector may be estimated, and changes in this orientation caused by a perturbing electromagnetic field may be estimated.

The real and imaginary components of an MR signal may also show changes. These components may be transformed into phase and magnitude measures according to common practice known to those skilled in the art, or they may be used directly in measurement, or they may be transformed into a coordinate frame to maximize the measured difference induced by an electromagnetic field (e.g. using principal components methods).

A challenge in the measurements just described is that many electromagnetic fields of interest may be very small 240 (e.g. in the range of 10-15 to 10-6 Tesla depending upon the magnitude of the field) relative to the field strength of measurement (e.g. 0.1 to 10 Tesla). Therefore, the resulting changes may be correspondingly small. In addition, a number of noise sources may produce changes in the phase, magnitude, orientation, or other characteristics of the MR signal. Some noise sources include fluctuations in the earth's magnetic field, fluctuations cause by the cardiac or respiratory cycle in subjects, fluctuations in the Bo field within an MRI scanner caused by the scanning hardware, fluctuations caused by applied gradient fields or radio frequency pulses or eddie currents, fluctuations caused by other electromagnetic sources in the immediate vicinity (e.g. lab electromagnetic noise). This process may be measured by presenting stimuli 270 at some time prior to or following an RF pulse 260 followed after at time TE by data acquisition.

Therefore, it may be desirable to compare the measured MR signal from a source location with the measured MR signal from a reference location, thereby performing differential measurements. The reference signal measurement may be made in a variety of ways. One method for measuring a reference signal is to use a second receive coil which measures an MR signal from a reference location, this location being susceptible to some of the same ‘common mode’ noise sources as the source location, but differentially susceptible to the signal of interest. For instance, in measuring an electrophysiological current of interest in a given source location, a reference location in the brain that is distant from the area of the electrophysiological current may be used that will be susceptible to much of the noise arising from sources other than the electrophysiological current of interest.

The reference signal may also be measured directly using alternate means to sensitively measure magnetic field strength, such as a magnetometer or gradiometer, e.g. a SQUID device, which is placed so as to provide a reference signal from a reference location. The reference signal may use measures similar to those employed in magnetoencephalography (MEG). The signal from the reference location may be subtracted from the signal at the source location in order to produce a differential signal. A number of methods have been developed for removing common mode noise from two or more electrophysiological signals in the context of current or voltage recording, as will be familiar to one skilled in the art.

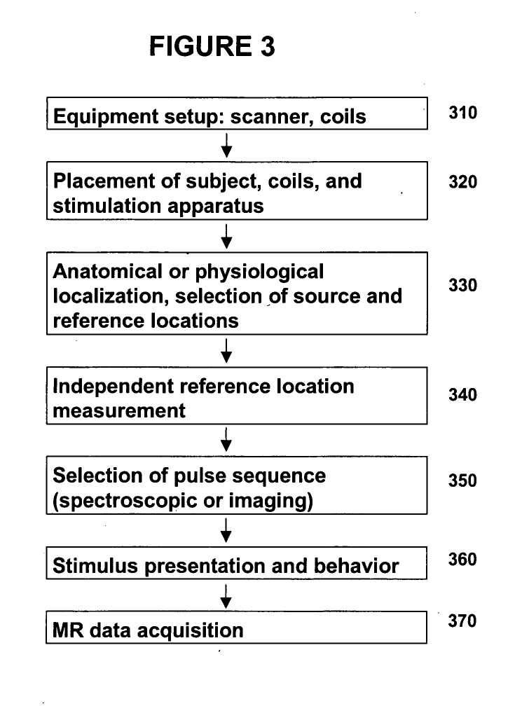

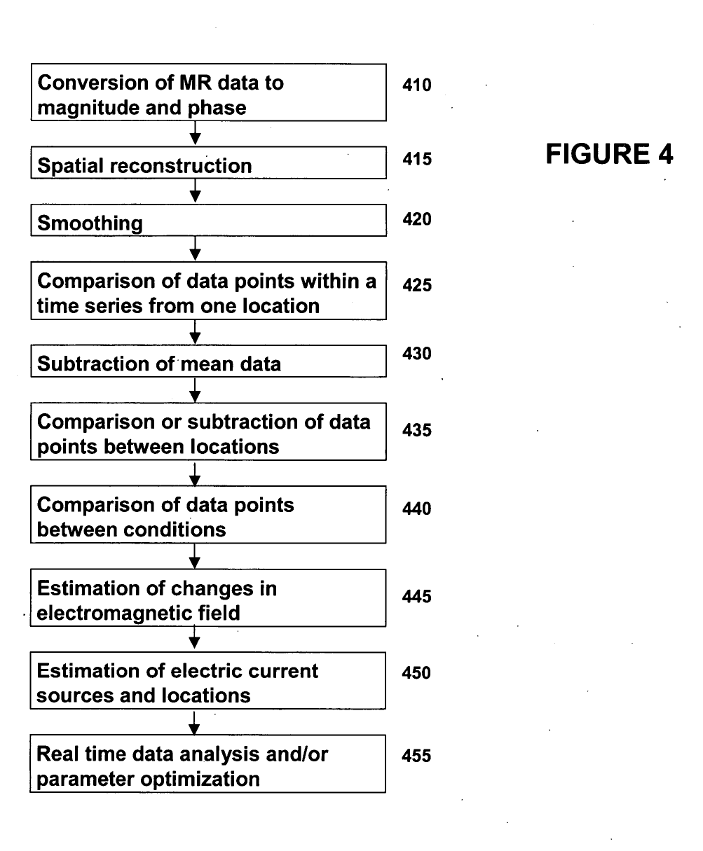

This method may be completed through the process described, and depicted in FIGS. 3 and 4.

Equipment Setup: Scanner Coils 310

In order to make MR measurements, an instrument for magnetic resonance imaging or spectroscopy may be employed. An example instrument is a 1.5 Tesla Signa MR imaging device produced by GE Medical, or MR measurement equipment manufactured by others. Methods for use of MR measurement devices and related imaging and spectroscopy devices are familiar to one skilled in the art, and are described in the relevant operator manuals. This method may be employed with an MR scanner of 0.1,0.5,1,1.5,3,5,7,20 Tesla or other values. The RF signal from the scanner may be transmitted using a combined transmit/receive coil, or the RF signal may be transmitted using a separate transmit and receive coil, a single transmit coil and multiple receive coils, or multiple transmit and receive coils. In one embodiment, a volume head coil is used, such as the GE Signa OpenSpeed Head coil or other quadrature birdcage head coil. In another embodiment one or more surface coils are used, or a phased array of coils is used. The RF may be transmitted from a body coil, and received through one or more surface coils.

Placement of Subject Coils, and Stimulation Apparatus 320

The subject to be measured may be placed within or adjacent to the measurement coils within the apparatus according to common procedures. In the case where more than one receive coil is being used, the coils may be placed parallel to one another, the coils may be placed so as to be orthogonal to one another, and the coils may be placed to be nearly co-planar. The coils may be placed so that they are parallel with the orientation of the electromagnetic field to be measured. The coils may also be placed so that they are orthogonal to the orientation of the electromagnetic field to be measured. The coils may also be placed so that one is parallel to the orientation of the electromagnetic field to be measured, and a second is orthogonal to the orientation of the electromagnetic field to be measured. The coils may also be placed so that they are obliquely oriented to the orientation of the electromagnetic field to be measured.

In addition, the axis of each coil may be positioned so as to be parallel or orthogonal to the primary magnetic field of the MR device. In one preferred embodiment, two 5″ surface receive coils are used, with one coil placed horizontally below the head of a human subject laying on their back, with a second coil placed orthogonally and lateral to the subject's head, with the subject laying on their back parallel to and within the bore of the scanner. Head restraint may be used to minimize subject movement, including the use of a bite-bar or cushions or other physical means designed to limit motion.

Anatomical or Physiological Localization, Selection of Source and Reference Locations 330

Anatomical localizer scans may be used to localize the regions from which measurements may be taken. Any of a variety of localizer methods may be used, such as a 3 plane localizer sequence available as part of GE Signa and other scanners. Localizers may include a variety of types of 2-D or 3-D anatomical scans such as T1-weighted scans, T2-weighted scans, proton-density-weighted scans, FLAIR images or other anatomical scans in common use currently or developed in the future.

Physiological localizer scans may be used to localize areas that are associated with activation of the brain caused by a given stimulus or task. Images of the activation level of brain regions associated with a given task or stimulus may be used independently, or superimposed upon anatomical images to allow localization to be based upon regions of defined activation. Physiological localization may use BOLD FMRI imaging, including substantially real time BOLD fMRI imaging. Physiological localization may be used to localize regions of the brain that are activated by a stimulus that will be measured using the MR measurement means described here. For example, the regions activated by a visual stimulus may be mapped using BOLD fMRI, and then one or more measurement locations selected to encompass regions activated by the visual stimulus, and then the same or a different visual stimulus may be used during measurement of MR perturbations caused by changes in electrophysiologically-based currents. BOLD FMRI has been well described in the literature and is familiar to one skilled in the art, e.g. US application 20020103429 Methods for physiological monitoring, training, exercise and regulation.

Once the target areas for measurement have been determined, the position of one or more source and/or reference locations for measurements may be defined, for example by using software by graphically selecting locations on images produced by the anatomical or physiological localizer scans. For example, using GE product PRESS sequences, it is possible to define a measurement volume using a graphic prescription. The positions may also be selected by designating locations relative to a known reference frame such as the scanner reference frame, or an anatomically-based reference frame or brain atlas. Saturation bands may be used to remove measured MR signal from some spatial regions. For example, the GE PRESS sequences allow for saturation bands to be removed from the selected area of measurement.

In one embodiment, a measurement voxel may be used that has two or more spatially discontinuous portions. It should be noted that this is a specialized use of the term voxel, in that a measurement voxel is a spatially defined volume that can have one, two or more spatially separated regions. MR measurements may be made using separate receive coils that are principally sensitive to each of the two or more discontinuous portions. In one example, one receive coil is placed nearer to one of the portions of the voxel, and a second receive coil is placed nearer to a second portion of the voxel, with each receive coil being differentially sensitive to the voxel portion that is nearer to it. In this way, the signals from the two receive coils may be used to measure signal from two different spatial locations. A voxel may be defined with two or more discontinuous regions by defining a continuous excitation voxel using spectroscopy software available on a scanner, and then applying spatial saturation techniques to reduce signal arising from a central section of that voxel, leaving two spatially separated regions that are not saturated. In one embodiment, the very specific saturation bands (VSS bands) used in conjunction with GE spectroscopy pulse sequences may be used. A long, rectangular voxel may be selected on a sagittal localizer slice, with the rectangle stretching from occipital cortex to frontal cortex, and then the majority of the central region of the rectangle may be saturated using a VSS band. In this way, one may create one source location in the occipital cortex that is sensitive to visual cortex currents, and may be principally received by a surface coil adjacent to the visual cortex, and a second reference location in the frontal cortex, that may be received principally be a surface coil adjacent to this location. In another example, a voxel may be selected that includes a portion of a nerve or fiber tract that is to be measured, with a second section of the voxel not including that nerve or fiber tract. This may allow measurement of current passage within the nerve or fiber tract, while excluding sources of noise.

In addition, MR imaging and chemical shift imaging sequences may be used that define a spatial grid of source voxels for measurement, and an additional reference location or spatial grid of reference locations. A second reference receive coil may be positioned so as to receive data only from one section of the MR image or chemical image. Saturation bands may also be placed so as to substantially remove MR signal from some voxels that are received strongly with the reference receive coil. In one example, a large spatial grid of MRI voxels or CSI voxels may be prescribed, such that some of the grid is primarily within the receive area of a source receive coil, and a second portion of the grid is primarily within the receive area of a reference receive coil. Saturation techniques may be applied so that the area of the grid within the receive area of the reference receive coil is removed, but for a small area or a single voxel.

The number of channels of data acquisition may be matched to the number of receive coils in use, and separate MR data (such as FID data) may be collected from each channel and used in further processing and measurement.

Independent Reference Location Measurement 340

The reference location may also be measured using means other than MR imaging. Other means for measuring the magnetic field from a reference location include the use of a sensitive magnetometer or gradiometer. This independent measurement device may provide an independent measurement of the magnetic field strength, gradient, or flux within the MR measurement instrument. Measures may also be used that depend directly upon the scanning hardware, such as measures of the current flowing in the magnet coils of an MR device. These independent reference location measurements may be used to correct for fluctuations measured at the source location that arise from these fluctuations measured at the reference location through the use of an independent reference measurement. Methods similar to those in use for magnetoencephalography (MEG) may be employed through the use of a one or more coil coupled to a SQUID in order to make very precise magnetometry or gradiometry measurements. In order to prevent interference between the MR measurements and reference location measurements, they may be made at separate but nearly coincident times, for example separated by 0.00001s, 0.0001s, 0.001s, 0.01s, 0.1s, 1s, 10s, 100s.

Selection of Pulse Sequence (Spectroscopic or Imaging) 350

A variety of MR pulse sequences may be applied for the measurement of signals from the source and reference locations. Imaging sequences may be employed to make measurements from a 1-D array of points, from a 2-D matrix of points, or from a 3-D volume of points, and using single-shot measurement or multi-shot measurement. Imaging pulse sequences may include spin echo and/or gradient echo imaging sequences, as has been described in Bodurka, J., and Bandettini, P. A., 2002. Toward direct mapping of neuronal activity: MRI detection of ultraweak, transient magnetic field changes. Magn Reson Med 47, 1052-1058, which is included herein by reference. In addition, this invention discloses that spectroscopic pulse sequences may be employed for the measurement of MR perturbations, including perturbations arising from electromagnetic fields. Spectroscopic pulse sequences may be employed for the measurement of MR perturbations, including perturbations arising from electromagnetic fields, that do not use imaging gradients, and that may measure data from a source volume repeatedly following an RF excitation to produce a free induction decay (FID). Imaging gradients may contribute to the noise in an MR signal (including phase noise and magnitude noise), due in part to variation in the imaging gradients presented. Therefore, using spectroscopic pulse sequences that do not use imaging gradients during readout is a mechanism of increasing the measurement power and sensitivity of the method by removing a noise source. Spectroscopic pulse sequences which may be used include PRESS sequences and STEAM sequences. Example spectroscopic pulse sequences that may be used in conjunction with a GE scanner include the press-csi and probe-p sequences, and the steam-csi and probe-s sequences.

Pulse sequences used to make MR measurements may include single or multishot acquisition methods, steady-state free precession methods, addition of diffusion sensitization gradients, magnetization transfer methods or other MR measurement pulse sequences already developed or that may be developed in the future. In addition, both gradient echo and spin echo methods may be employed. In one embodiment, one or more refocusing RF or gradient pulse may be used to allow measurement of larger MR signals at greater time points from an initial RF shot. An example is using spin echo methods, such as the formation of Hahn echoes, to overcome signal decay. In addition, imaging sequences may use a variety of readout patterns, including spiral in, spiral out, spiral in/out, and echo planar imaging patterns.

In one embodiment, press imaging is performed using a GE signa scanner, with the following parameters: Plane=sag, Mode=MRS, Imaging options=EDR, TE=30 msec, TR=1000 msec, FOV=24 cm, Nex=1, F-dir=S/I, bandwidth=2000, points measured=512, voxel size=0.1,2.,5,1,2,5 cm\hat{ }3.

Stimulus Presentation and Behavior 360

MR measurements may be made during, shortly following, or shortly preceding the presentation of a stimulus that may be expected to induce neural activation that in turn induces a current or electromagnetic field that is to be measured. A stimulus that elicits activation in the brain may include a visual image or sequence, an auditory sound or sequence, a tactile sensation, an electrical stimulus to either a peripheral location or directly to the central or peripheral nervous system, a pharmacological or other physiological stimulus, or other perceptual stimuli or instructions. In addition, currents may be induced accompanying electrophysiological events associated with cognitive or behavioral processes such as the performance of a mental task, or the performance of a movement. The MR signals measured during, following, or preceding the presentation of a stimulus or performance of a task may be compared with one another in order to determine the effect of the stimulus or task on the measured currents. The period of time between the presentation of the stimulus and the initiation of MR measurements may be +/−1 ms, +/−2 ms, +/−5 ms, +/−10 ms, +/−20 ms, +/−40 ms, +/−50 ms, +/−100 ms, +/−250 ms, +/−1000 ms, +/−10000 ms, where +/− indicates that the stimulus may either follow (+) or precedes (−) the initiation of MR measurement by the specified time. A separate stimulus may be presented immediately preceding or following the MR signal measurement period following each ‘shot’ of the scanner.

In one embodiment, multiple MR measurements are made at a fixed time following successive, repeated single RF shots with a temporal delay (TR) between RF shots. A brief (e.g. 1, 5, 10, 50, 100 ms) visual stimulus is presented to the subject immediately preceding or coincident with the readout period following some of the RF shots. This visual stimulus is expected to produce activation in the source location being measured, for example the visual cortex or optic nerve. This stimulus may be presented using a reverse projection screen with commonly used methods for stimulus presentation. The stimulus may be precisely time-synchronized following the time of the RF shot. On other RF shots, no stimulus is presented, or the stimulus is presented some time after the measurement is initiated or completed. The MR measurements immediately following the stimulus, during the period when electrophysiological activity is taking place, may be compared with MR measurements prior to the presentation of a stimulus, or when no stimulus is presented, and when less or no stimulus-evoked electrophysiological activity is taking place. By subtracting the MR measures in the condition when the stimulus is presented form the MR measure in the case when the stimulus is not presented, the effect of the stimulus may be observed. In addition, by making successive MR measurements at different time points after the presentation of the stimulus, a time course of the response to the stimulus may be generated.

In addition, as an additional means of measuring the MR signal at different times relative to the time of the stimulus onset, the time of the stimulus relative to the RF shot may be changed for different individual RF shots, and the MR signal measured at one or more fixed times relative to the RF shot. Thereby, it may be possible to estimate the average phase and magnitude of the MR signal at different times relative to the onset of the stimulus.

The MR signals used may be signals from a single source location, they may be difference signals computed by subtracting a reference location from a source location, or they may be imaging or volume signals that correspond to multiple spatial locations or differences between multiple spatial locations and a reference signal or location.

The same types of measurements just described for measuring electrophysiological responses resulting from a stimulus may be employed to measure electrophysiological responses resulting from the performance of a task, or the chemical, electrical, or other stimulation of electrophysiological activation, including activation using transcranial magnetic stimulation. In addition, in the case of stimulation methods that involve the application of electric currents or electromagnetic fields, the current or field applied may be measured using the methods described, in addition to the resultant electrophysiological phenomena.

In the case of measurements involving an electrical system such as an artificial circuit, measurements may be made comparing the case where current is applied through the system vs the case when less or no current is applied. Measurements may also be made comparing the case where the system is in one functional state (e.g. turned on or conducting a process) vs the case when the system is in a different functional state (e.g. turned off or conducting a different process). This allows for the MR measurement of the perturbation of the electromagnetic field due to the current conducted by the circuit.

MR Data Acquisition 370

MR data may be collected from the sample. This data may be processed as described herein. Additional processing steps and applications may be as described in US application 20020103429 Methods for physiological monitoring, training, exercise and regulation, including using any computations described in the section performing computations on images using analysis and control software. In particular, all analyses described in the sections entitled ‘Processing of scan data into images and metrics in substantially real time’ and ‘Performing computations on images using analysis and control software’ may be applied.

MR data may be collected from one or more receive elements, for example in order to measure the differential contribution of a perturbation upon the volume measured by each receive element. MR data may be collected at one or more time points relative to the time of application of RF energy. MR data collection may involve the application of changing magnetic gradients, such as imaging gradients, or may be made in the absence of such gradients. MR data may involve the measurement of multiple components at each measurement point, such as the measurement of real and imaginary components of an MR signal or phase and magnitude components of an MR signal. Using a pulse sequence, the collection of data may begin at a time after the excitation pulse (TE) of approximately 0, 0.1, 1, 5, 10, 20, 30, 50, 100, or 500 ms, and may take place for approximately 0.1, 1, 5, 10, 20, 50, 100, 200, 500, 1000, or 2000 ms at a sampling rate of about 0.1, 0.5, 1, 2, 5, 10, 20, 100 kHz. Collection of data from more than one location may take place substantially simultaneously, or separated by about 0, 0.1, 1, 5, 10, 20, 30, 50, 100, 500, 1000, or 10000 ms. This may allow for the differential measurement of MR signals from two or more spatial locations measured at substantially the same time, or at times separated by about 0, 0.1, 1, 5, 10, 20, 30, 50, 100, 500, 1000, or 10000 ms.

It is here disclosed that in order to measure the time course of the perturbation of an electromagnetic field with a rapid sampling rate, it is possible to collect multiple data points following a single RF excitation during the time evolution of a free induction decay (FID), and use this data to infer the time course of change of the electromagnetic field. This may be accomplished, for example, using spectroscoptic measurement pulse sequences such as PRESS. It is also possible to continuously monitor the MR signal through time if additional, intervening RF pulses or gradient pulses are employed, for example in the case of SSFP, or when using an additional RF or gradients to refocus an MR echo. Since neuronal currents evolve over a time course in the range of 1 to 500 ms, it is possible to continuously record MR signals over a corresponding time period of about 0.1, 1, 5, 10, 20, 50, 100, 200, 500, or 1000 ms by making repeated measurements, and then use the MR data to make inferences about the time course of perturbations in the magnetic field, and thereby to make inferences about the time course of neuronal currents.

As one example of the collection of MR data, real and imaginary components of the MR signal may be repeatedly measured simultaneously from each of two receive coils at 1 ms intervals from 30-130 ms following an RF pulse, using a PRESS spectroscopy sequence with a TE of 30 ms. Therefore, 100 real/complex data pairs may be recorded substantially simultaneously from each of the two coils following the RF pulse. These two coils may be positioned adjacent to the occipital and frontal surfaces of the head of a subject. This process may then be repeated many times, with a delay between RF pulses of Is (TR). For some fraction of the RF shots, a stimulus such as a flashed visual stimulus designed to evoke neural current may be presented to the subject coincident with the initiation of data recording. On other RF shots, a stimulus may not be presented.

Post-Processing of MR Data

This invention discloses the use of differential amplification of MR phase and magnitude signals. Differential amplification may include computing a difference of an amplified MR signal measured from two different receive elements within an MR instrument. Differential amplification may also include the computation of a difference of two MR signals measured from the same receive element at two different time points separated by a short (about 0.0001, 0.001, 0.01, 0.1, or 1 s) interval to remove slow signal components not due to the electromagnetic component being measured. Differential amplification may also include the filtering of a timecourse of MR signals measured a single same receive element to remove slower or faster signal components not due to the electromagnetic component being measured. Differential amplification may also include the computation of a difference or the filtering of MR signals measured from two or more different receive element at two or more different time points separated by short (about 0.0001, 0.001, 0.01, 0.1, or is) intervals to remove slow signal components not due to the electromagnetic component being measured. These and other computations may be achieved through differential post-processing of MR data.

Differential post-processing of raw data may include a series of components whose descriptions follow. In this post-processing, any of the steps may be left out of the analysis, either individually, or in combination. The analysis steps may also be performed in different orders. Differential post-processing analysis may be performed on single time point data or on time series data from a single measurement location. Differential post-processing analysis may be performed on single time point or time series data from more than one measurement location. Differential post-processing analysis may be performed on single time point or time series data of the differences between measurements between two locations. Differential post-processing analysis may be performed on single time point or time series data from a 1-D, 2-D or 3-D array of values corresponding to a measurement line, plane, or volume. Differential post-processing analysis may be performed on single time point or time series data of the difference between a 1-D, 2-D or 3-D array of values and values from a reference location. Time series MR data may correspond to a free induction decay (FID). Additionally, reference location data may be taken from an independent method of measurement, such as a magnometry or gradiometry measurement, rather than an MR measurement, and used in the following analysis steps.

Conversion of MR Data to Magnitude and Phase 410

As a component of differential post-processing, raw MR data points that are collected in terms of real and imaginary parts may be transformed into phase angle and magnitude measures. In this way, phase may be separated and changes in phase may be measured in isolation from magnitude, and magnitude changes may be measured in isolation as well. Alternatively, data points may be transformed into a different basis that has been shown to maximize the observed difference between two conditions (e.g. stimulation vs. no stimulation).

Spatial Reconstruction 415

As a component of differential post-processing, in the case where magnetic resonance imaging is used, data may be spatially reconstructed from raw k-space data into image space or volume data using standard methods, e.g. Glover, G. H., and Lai, S., 1998. Self-navigated spiral FMRI: interleaved versus single-shot. Magn Reson Med 39, 361-368; Lai, S., and Glover, G. H., 1998. Three-dimensional spiral FMRI technique: a comparison with 2D spiral acquisition. Magn Reson Med 39, 68-78. In the case where chemical shift imaging is used, data may be spatially reconstructed from raw data into space or volume to data, e.g. time series data (e.g. FID data). The spatial reconstruction process may produce magnitude and phase data for each measured point from raw k-space data. In the case where a free induction decay (FID) is used without imaging gradients, this step may be omitted. In the case where chemical shift imaging is used, spatial reconstruction may take place to produce an FID for each spatial location as is typical for CSI data.

Smoothing 420

As a component of differential post-processing, collected time series MR data from any location may be smoothed to remove high frequency noise, or data may be bandpass filtered. For example, data may be filtered to remove components with frequencies higher than 100 Hz.

Comparison of Data Points within a Time Series from One Location 425

As a component of differential post-processing, different time points from time series MR data may be compared. In one embodiment, for each RF shot, a time series vector of measurements are reconstructed for both phase and magnitude, and the first value in each time series (or a value at some time point other than the first time point within each series) is subtracted from each other value in the time series to form a new time series. In this way, any noise in the start point in the series is removed, and more sensitive measures may be obtained that may be less sensitive to jitter in the start point. Further, the mean, linear or higher-order trends may be removed from the time series data measured from any location.

Subtraction of Mean Data 430

As a component of differential post-processing, multiple measurements may be made from each measured location, such as by using repeated RF shots and measurements. A single value selected for the data following one RF excitation may be subtracted from each data point so that remaining analysis is focused on the trial-to-trial differences in measurements. The mean of all data points from a location for a single RF shot may be subtracted from each data point so that remaining analysis is focused on the trial-to-trial differences in measurements. This subtraction of data from different time points may be made possible through the acquisition of a full free induction decay (FID) following an RF excitation, a time series of data from a single location, rather than using the conventional imaging method of measuring a single complex data pair representing a single time point for each spatial location after an RF excitation. This method may be used to remove factors affecting the MR signal that are common across all trials, such as eddie currents, and to bring out factors that are different on different trials, such as on trials where a stimulus was presented vs. trials where a stimulus was not presented. Rather than using an overall mean, a mean may be subtracted from each trial that is only a mean of trials that took place at nearby times, such as through the subtraction from each trial of the mean of all trials within a specified number of trials from the specified trial. Trials may also be clustered into groups, and the mean of each cluster subtracted from each trial within the cluster. Trials may be clustered into groups by selecting a value n, and then clustering trials so that each successive n trials form a new cluster, and have the mean of that cluster subtracted. The value of n may be set to equal a repetition cycle of a number of conditions that are successively used, such as using clusters of three when three different stimulation conditions are repeated in sequence.

Comparison or Subtraction of Data Points 435

Comparison or Subtraction of Data Points Measured from Different Locations using a Single Receiver

To compute a differential measure, MR data may be compared or subtracted between different spatial locations measured using a single receiver through the use of MR imaging. MR imaging allows for the measurement of MR signals from multiple locations using the same receiver, for example by the use of imaging gradients. Following the computation of an MR signal from more than one spatial location using a single receiver, the MR signals from different locations may be compared. MR signals from different locations using a single receiver may be compared by subtraction of the complex values. MR signals from different locations using a single receiver may be compared by subtraction of the phase components, or of the magnitude components. MR signals from different locations using a single receiver may be compared by subtraction of the results of transformation of the initial MR data, such as transformation into a different coordinate basis than the original real/imaginary basis or the phase/magnitude basis. MR signals from different locations using a single receiver may be compared through the comparison of individual MR measurements, or through the comparison of a full time series of MR measurements.

Comparison or Subtraction of Data Points Measured from Different Locations using More than One Receiver

To compute a differential measure, MR data may be compared or subtracted between different spatial locations measured using more than one receiver, with or without the use of MR imaging. The use of more than one receiver may allow for separate measurements from different spatial locations, and may allow for separate measurements from different spatial locations to be made without the use of imaging gradients. Following the computation of an MR signal from more than one spatial location using more than one receiver, the MR signals from different locations may be compared. MR signals from different locations using a single receiver may be compared by subtraction of the complex values. MR signals from different locations using more than one receiver may be compared by subtraction of the phase components, or of the magnitude components. MR signals from different locations using a single receiver may be compared by subtraction of the results of transformation of the initial MR data, such as transformation into a different coordinate basis than the original real/imaginary basis or the phase/magnitude basis. MR signals from different locations using more than one receiver may be compared through the comparison of individual MR measurements, or through the comparison of a full time series of MR measurements.

Comparison or Subtraction of Data Points Measured at Different Time Points

To compute a differential measure, MR data may be compared or subtracted that has been collected at different time points, separated in time by about 0.01, 0.1, 1, 5, 10, 100, 1000, or 10000 ms. To compute a differential measure, MR data may be compared or subtracted that has been collected at different time points from the same spatial location and the same receiver, separated in time by about 0.01, 0.1, 1, 5, 10, 100, 1000, or 10000 ms. To compute a differential measure, MR data may be compared or subtracted that has been collected at different time points from the same spatial location and different receivers. To compute a differential measure, MR data may be compared or subtracted that has been collected at different time points from the different spatial locations and the same receiver. To compute a differential measure, MR data may be compared or subtracted that has been collected at different time points from the different spatial locations and different receivers. MR signals from different time points may be compared by subtraction of the complex values. MR signals from different time points may be compared by subtraction of the phase components, or of the magnitude components. MR signals from different time points may be compared by subtraction of the results of transformation of the initial MR data, such as transformation into a different coordinate basis than the original real/imaginary basis or the phase/magnitude basis. MR signals from different time points may be compared through the comparison of individual MR measurements, or through the comparison of a full time series of MR measurements.

Additional Differential Measures

Two or more measures may be compared in a variety of ways. For example, in order to produce a differential measure two MR measurement values may be subtracted. This comparison may be made of a single pair of MR measurement values or a single pair of time-series of MR measurement values. Additional methods familiar to one skilled in the art may also be used to form differential signals. In one example, a differential measurement between a source and a reference signal may be computed using a difference from the prediction of a statistical model based upon the reference data. This statistical model may include a linear correlation model, a higher order correlation model, a general linear model, a principal components model, an independent components model or other statistical models familiar to one skilled in the art. For example, an average linear correlation model may be computed between the values from a reference location and the values from a source location. The resultant model reflects the correlated or common-mode components between the two locations. Therefore, the model may be used to predict the values at the source location based upon the values at the reference location. Remaining, unpredictable variance at the source location will reflect uncorrelated noise, and independent signals. Therefore, an estimation of the independent signal at the source location may be computed as the residual variance after the model-based prediction formed using the values from the reference location has been removed. In one example, the common-mode signal may be partialled out from the source location using statistical regression methods, such as using a general linear model, leaving a residual signal that corresponds to the signal at the source location that cannot be ascribed to variance at the reference location. This process may be performed for each time point in a time series separately, or in conjunction. Principal components methods may also be used to separate out one or more components due to the electromagnetic signal vs components due to noise.

Comparison of Data Points Between Conditions 440

The resultant single time point data or time series data may be compared between different measurement conditions in order to make an estimate of the effect of the different conditions. The data collected following RF shots when a stimulus was presented, behavior took place, or current was injected may be compared with data collected following RF shots when there was no stimulus, behavior or current, or a different stimulus, behavior, or electric current was used. This allows an estimation of the effect on the signal of the presented stimulus, behavior, or electric current. One type of comparison is a subtraction of the time series differential MR phase signal (the time course of MR phase at a source receive element minus the time course of MR phase at a reference receive element) observed following a stimulus from time series differential MR phase signal observed when there was no stimulus. One type of comparison is a subtraction of the time series differential MR magnitude signal (the time course of MR magnitude at a source receive element minus the time course of MR phase at a reference receive element) observed following a stimulus from time series differential MR phase signal observed when there was no stimulus. It should be understood to one skilled in the art, that this method may be used to compare among any different types of conditions that may be induced or observed in the subject being measured.

Estimation of Changes in Electromagnetic Field 445

The magnitude of a difference in electromagnetic field between two conditions may be estimated by measuring the amount of change in the MR signal between the two conditions, and correlating this with computed or observed perturbations caused by electromagnetic fields of know magnitude. The observed perturbations may have been measured previously using a form of standard such as a ‘current phantom’, as disclosed here. A current phantom may be a vessel with a means running through it that can carry currents of known values, and that can be used to measure the resultant change in MR values caused by those currents. In one embodiment, the change in MR phase or magnitude that takes place over the time period measured in a time series may be converted into a change in associated resonance frequency. The change in MR phase or frequency may be used to compute a change in electromagnetic field using the Larmour equation, as will be familiar to one skilled in the art.

Estimation of Electric Current Sources and Locations 450

Electric currents produce electromagnetic fields following known and lawful behavior, such as that described by the Maxwell equations. The direction of current flow may be estimated from estimates of perturbations of the electromagnetic field calculated using this method. The data of electromagnetic field values at one or more spatial locations observed using the method described here may be used as input into methods for current source density estimation, or dipole localization, in order to produce estimates of current direction, magnitude, and location, or dipole localization. Methods for dipole localization and electric current source localization have been well described in the literature, for example in: Miga, M. I., Kerner, T. E., and Darcey, T. M., 2002. Source localization using a current-density minimization approach. IEEE Trans Biomed Eng 49, 743-745; Schimpf, P. H., Ramon, C., and Haueisen, J., 2002. Dipole models for the EEG and MEG. IEEE Trans Biomed Eng 49, 409-418; Yoshinaga, H., Nakahori, T., Ohtsuka, Y., Oka, E., Kitamura, Y., Kiriyama, H., Kinugasa, K., Miyamoto, K., and Hoshida, T., 2002. Benefit of simultaneous recording of EEG and MEG in dipole localization. Epilepsia 43, 924-928. The data measured here a field perturbations may be input into models for current source localization in a similar fashion to the data used from MEG recordings, as will be familiar to one skilled in the art.

Substantially Real Time Data Analysis and/or Parameter Optimization 455

This invention discloses the use of substantially real time MR imaging, substantially real time MR spectroscopy, and substantially real time chemical shift imaging, as well as the use of these methods in the measurement of MR perturbations, including perturbations arising from changes in magnetic field strength or electric current. Some or all of the analyses described here may be achieved in substantially real time. Substantially real time analysis means analysis that takes place within about 0.001, 0.01, 0.1, 1, 10, 100, or 1000 seconds of the acquisition of each data point following an RF shot. Once data has been analyzed in substantially real time, the results of this analysis may be used to optimize the parameters of the measurements being made. For example, the many parameters used in controlling MR data acquisition may be automatically or manually adjusted in order to produce an increase in the resultant MR signal magnitude or phase, a decrease in the variance of the signal, or an increase in the magnitude or decrease in the variance of the measured estimated change in electromagnetic field caused by a stimulus, behavior, or electric current. Automatic adjustment may be made using a computer-controlled feedback loop and appropriate control software. Some of the parameters that may be optimized using this data include TE, TR, spatial size or location of each measurement location, RF frequency, linear or higher-order shim currents, transmit and receive gains, numbers of excitations, water or fat suppression, inversion of RF pulses, magnetic field gradient magnitudes or slew rates, or other parameters that may be adjusted to optimize MR measurements.

For example, the methods disclosed here may be used to measure the current induced by a stimulus using a given set of parameters for making MR measurements. The MR measurement parameters may then be changed, and an additional measurement of current may be made. Then, the parameters may be further adjusted to optimize the signal to noise ratio of the current being measured vs. sources of noise. The time between RF excitations, TR, influences the magnitude and SNR of the signal, and also the amount of data that is collected. Therefore, MR data may be collected and processed as disclosed at multiple values of TR in order to determine which value of TR produces the most reliable estimate of a perturbation of the electromagnetic field in a given amount of time. This process of altering the TR to achieve an optimal result may be automated. The echo time, TE, may also be modified in order to optimize the magnitude of the current measured vs the noise.

Use of Signals in Training of Subjects and for Other Purposes

Brain activation information derived from the invention disclosed here may also be used for training of subjects as disclosed in US Appl. Publ. No. 20020103429 Methods for physiological monitoring, training, exercise and regulation. For example, information measured as described here may be used as activation information for a region of interest for training.

Differential MR Recording, which May use a Separate Transmit and Differential Receive Coils

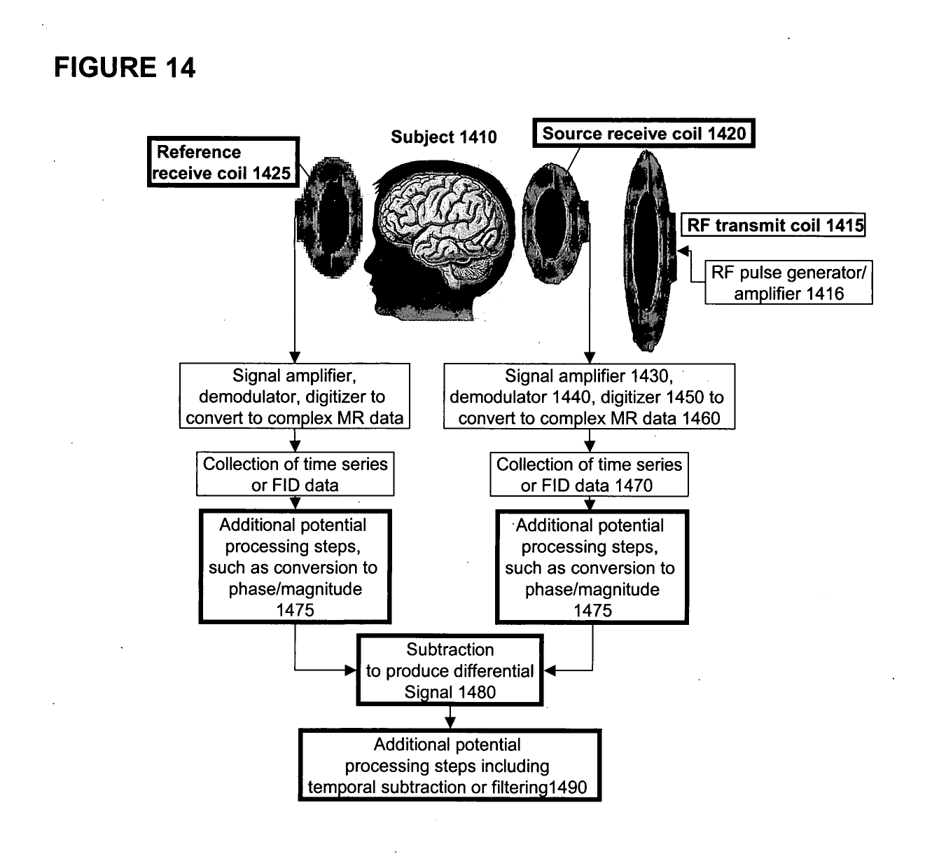

FIG. 14 depicts a diagram of the methods and equipment involved in the differential measurement of MR signals. As depicted, RF excitation is delivered by a transmit coil, and received by two separate receive coils. It is also possible for a single coil to both transmit and receive RF energy. The signals from two or more receive coils may then be processed, and compared, for example to form a differential signal, as depicted in the figure, and further processing may additionally be carried out.

Use in Measurement of Specific Brain Areas

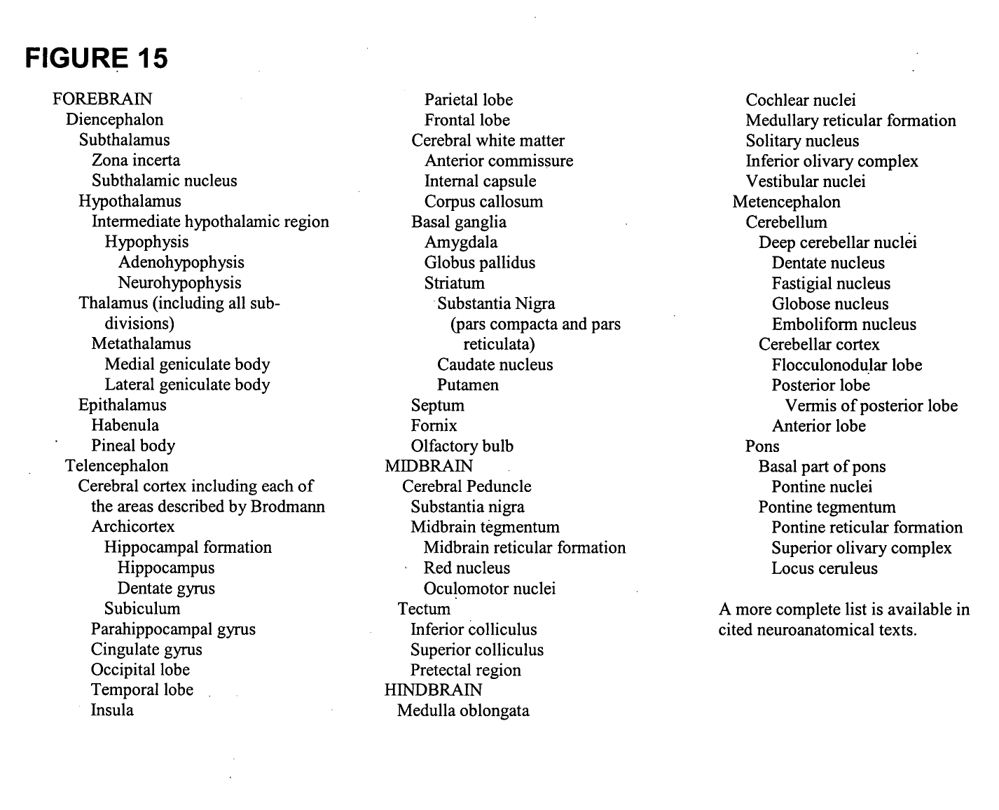

The invention described here may be applied to the measurement of perturbations in magnetic fields arising in a variety of specific brain areas. The perturbations may be used to infer electrical activity emanating from neuronal or physiological processes taking place within specified brain areas. A partial list of brain areas is presented in FIG. 15. In order to measure currents associated with neuronal processes in a certain brain area, measurements may be made from a voxel corresponding to the brain area. This may take place through the graphical prescription of a target voxel corresponding to the target anatomical structure, using anatomical localizer images, or functional localizer images to designate the position of the anatomical structure. In the case of functional localization, the area to be targeted for measurement may be selected based upon the activation observed in the area, for example using substantially real time fMRI.

Use in Diagnosis

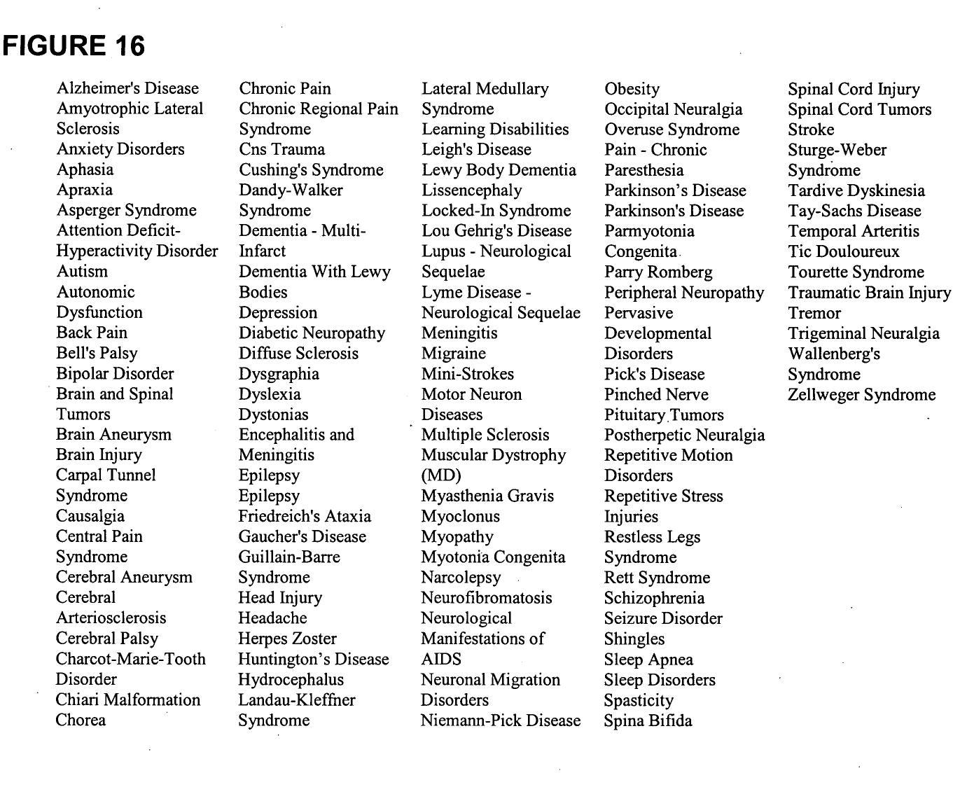

The invention described here may be applied to the diagnosis of functional abnormalities or diseases. A functional abnormality or disease state involving the central nervous system may be associated with an altered pattern of electrophysiological activity. For example, in the case of an epileptic focus, there may be an increase in electrical activation emanating from brain tissue. In the case of a brain area involved in an injury or compromised by degenerative or other central nervous system disease, there may be a decrease in electrical activity emanating form the brain tissue. The electrical activity may be either spontaneous activity, or may be activity elicited by a particular stimulus, or by symptom provocation. Therefore, the invention described here may be used to diagnose abnormal functioning of a brain region. In addition, by comparing the functioning of a brain region using this method between an individual and both a healthy population or a population with a particular disease condition, it may be possible to diagnose the presence of a given CNS disease condition. Examples of CNS disease conditions that may be subject to diagnosis in this fashion are included in FIG. 16.

Methods are provided for diagnosing and treating an area of the brain that has been compromised by a stroke or other cerebrovascular or other neurologic injury. According to these methods, the diagnosis may be conducted in combination with performing measuring MR perturbations in brain regions of interest according to the present invention.

When a subject has had a neurologic injury, such as a stroke or other cerebrovascular or other neurologic injury, mapping may be performed to determine what regions of the brain have been compromised by the injury. The extent or progression of the damage may also be evaluated. For example, anatomical mapping can provide one indication of the areas compromised by a cerebrovascular accident. A second indication of the areas of damage or partial disfunction may be provided by performing physiological measurements of brain activity through the methods provided here. In order to achieve this, the physiological activation patterns in subjects are measured, such as by measurements according to the present invention.

Mapping may be used as a diagnostic tool to detect areas that have been injured. The diagnostic method may simply include measuring an activation pattern of a subject while the subject is presented with one or more stimuli and/or engaged in one or more behaviors that are designed to activate regions of interest of the brain, including regions thought to be potentially compromised by the neurologic injury. The activation may then be compared with activation when the subject is in a rest state in order to determine a background level of activity. The activation may also be compared with the activation observed in an unimpaired subject performing a comparable task.

Regions where no activation is observed can be surmised to be compromised zones. Regions where only low levels of activation or other abnormal activity metrics are observed in comparison with healthy subjects undergoing the same tasks may be surmised to be partially compromised.

The variance measured in the activity level or other activity metric during a rest or task condition for any brain voxel can be used as an indicator of the state of the corresponding neural tissue. Voxels with very little of the normally observed fluctuation in the background level of activity can be surmised to be affected or compromised by neurologic injury. This may allow an automatic mapping process of the level of signal fluctuations to take place that may provide an indication of the regions affected by a given injury, disease or condition. In addition, this mapping may be used to measure the level of fluctuation in different brain areas within a restricted temporal frequency band, such as to measure the corresponding level of brain activation in the alpha range, beta range, gamma range, delta range, theta range, or other frequency bands.

Triggering Scanning by an External Event

The timing of MR measurement initiation may be triggered by the time of an external event. In one example, MR measurement initiation may be triggered using methods available on current MR scanners such as cardiac or respiratory triggering. The time of initiation of MR measurement using this method may take place at a substantially similar time point within the cardiac cycle. The time of initiation of MR measurement using this method may take place at a substantially similar time point within the respiratory cycle. The time of initiation of MR measurement using this method may take place at a substantially similar time point relative to the presentation of a stimulus. The time of initiation of MR measurement using this method make take place at a substantially similar time point to the production of a behavior such as a movement recorded by a recording device. The time of initiation of MR measurement using this method may take place at a substantially similar time point relative to the time of presentation of an electric current or stimulus.

Triggering an External Event by Scan Initiation

The timing of MR measurement initiation may trigger the time of an external event. In one example, MR measurement initiation may trigger the time of initiation of the presentation of a stimulus. The time of initiation of MR measurement using this method may take place at a substantially similar time point relative to the time of presentation of an electric current, magnetic or other stimulus. Triggering may be used to trigger the presentation of a stimulus, behavioral instruction or current relative to the time of initiation of MR measurement. The relative time of presentation of the stimulus compared with the time of initiation of RF excitation or MR data readout may be precisely controlled. The time of stimulus presentation before or after initiation of data readout may be, for example, about 0, +/−1, +/−2, +/−5, +/−10, +/−50, +/−100, +/−1000, or +/−10000 ms.

Ionic Currents

The invention disclosed here may be used to measure ionic currents. Ionic currents include ionic currents arising from physiological sources, as well as ionic currents arising from artificial processes including dissolution, membrane barrier permeation, or ionic conduction.

Use with Multi-Voxel MR Time Course Measurement or Chemical Shift Imaging

The invention disclosed here may be used to simultaneously measure the time course of magnetic field perturbations at each of a 2D array of locations; or at each of a 3D array of locations, using methods related to chemical shift imaging, CSI, or spatially-resolved multi-voxel spectroscopy. In this way, it is possible to measure the time course of the perturbation of a magnetic field at multiple different spatial locations within an object simultaneously. This may be accomplished without the use of imaging gradients during the readout phase of data acquisition. The time course of MR data, including phase and magnitude data, may be measured from multiple locations in space by the use of phase encoding during excitation, analogous to the method used to achieve spatial separation for CSI imaging using phase encoding. The resultant MR data may then be processed using FFT methods familiar to one skilled in the art to produce a separate average MR time course signal for each spatial location within the 2D or 3D area being measured. This process of measuring MR timecourse data from more than one spatial location simultaneously may be performed using a single receive element. This process may be performed using multiple receive elements. This process may be performed using a differential signal computed from more than one receive element.

In order to measure the perturbation in a magnetic field resulting from a stimulus or other event, the average time course of the MR signal from multiple spatial locations may be measured in the presence of the event, and in the absence of the event, and these two conditions may be compared. This may produce an estimate of the effect of the event on the perturbation of the magnetic field at multiple spatial locations. This may also produce an estimate of underlying currents at multiple locations that would lead to the observed perturbations of the magnetic field. This process may be performed using interleaving of trials with different stimulus conditions. In one example, using an 8×8 phase encoded multi-voxel spectroscopy (PRESS-CSI) grid, 64 excitation/readout events would be required to map out the time course of MR signal at each of the 64 voxels without a signal induced by a stimulus present. An additional 64-excitation/readout events would be required to map out the time course of MR signal at each of the 64 voxels with a signal induced by a stimulus present. It is possible to make these two sets of 64 measurements, perform the 2D FFT to produce two 8×8 sets of time course data, and then compare the data from each location. The MR phase and magnitude from each location may be compared separately. The phase data, for example, may be used to infer the shift in the magnetic field corresponding to the applied current at each location. However, since conditions may have slowly changed between the first set of 64 measures and the second set of 64 measures, due to other factors such as temperature, subject movement, or others, it may be desirable to interleave the stimulus/no stimulus trials within each of the two sets of 64 measures, and then re-sort the data upon completion to produce two resulting sets of 64 measures, one taken from trials when stimuli were present, and the other taken from trials when stimuli were absent. For example, a 64-excitation/readout set (A) may be collected with stimuli presented on the even numbered excitations, and then a second 64-excitation/readout set (B) may be collected with stimuli presented on the odd numbered excitations. The data from (A) and (B) may then be re-sorted into one set of data corresponding to 64 excitation/readout datasets with stimulus present, and another set corresponding to 64 excitation/readout datasets with the stimulus absent. These may then be reconstructed into two 8×8 sets of MR time course data. The data from these two 8×8 sets may then be compared to observe the perturbation in the magnetic field associated with the stimulus.

Reference Correction of Imaging, CSI or other Multi-Voxel Readout MR Data

The invention disclosed here may be used to correct imaging data for phase or magnitude variations that take place over the course of imaging readout, using either a single receiver or multiple receivers, and using differential or non-differential MR measures. In some instances, a noise source may change the phase and/or magnitude of an MR signal at both source location and a reference location in a correlated way. This correlated noise may also evolve over the course of a measurement readout period. This may be corrected for using data from a reference or source location. This process may be used to produce differential measures, for example MR FIDs, MR images or CSI images that reflect differential measures viz. a reference location.

In order to perform a correction, the value of phase and magnitude may be measured from a source location, and also from a reference location. The measures from the reference location may be measured using MR measurements. The measures from the reference location may be measured using non-MR measures. The measures from the reference location may include measures of the magnetic field made by a device capable of making such measures, for example a gradiometer or magnetometer. The initial value or slope for measures from a source or reference location may be used to correct for noise in the source location.

Values of one or more source location pre-measures Sₚᵣₑ 532, and/or source measures S_{1-N} 534, and/or source post-measures Sₚₒₛₜ 536 maybe collected on each of a number of trials. If imaging gradients are being used, the pre and post measures may be collected with magnetic gradients selected so that they correspond to values for the same point in k-space, or the same location. The average value of the reference location pre-measures <Sₚᵣₑ>, measures <S_{1-N}>, and post-measures <Sₚₒₛₜ> may be computed from a number or trials. For each trial, a deviation from this average may be computed for each value:
Sₚᵣₑdeviation=Sₚᵣₑ−<Sₚᵣₑ>
Sₚₒₛₜdeviation=Sₚₒₛₜ−<Sₚₒₛₜ>
S_{trend}deviation=S_{trend}−<S_{trend}>

Also, a reference linear trend S_{trend} 538 may be computed as the rate of change of the measure during the time interval between Sₚᵣₑ and Sₚₒₛₜ. A deviation of the trend for each trial from the average trend may also be computed. Each of these values may be collected or computed either as a complex value, or after transformation into separate phase and magnitude components or using another basis. The separate Sₚᵣₑ and Sₚₒₛₜ and S_{trend} components may be computed separately for phase and magnitude.

Values of one or more reference location pre-measures Rₚᵣₑ 542, reference location measures R_{1-N} 544, and reference location post-measures Rₚₒₛₜ 546 may be collected on each of a number of trials. If imaging is being used, the pre and post measures may be collected with magnetic gradients selected so that they correspond to values for the same point in k-space, or the same location. The average value of the reference location pre-measures <Rₚᵣₑ>, reference location measures <R_{1-N}>, and reference location post-measures <Rₚₒₛₜ> may be computed. For each trial, a deviation from this average may be computed for each value:
Rₚᵣₑdeviation=Rₚᵣₑ−<Rₚᵣₑ>
Rₚₒₛₜdeviation=Rₚₒₛₜ−<Rₚₒₛₜ>
R_{trend}deviation=R_{trend}−<R_{trend}>

Also, a reference linear trend R_{trend} 548 may be computed as the rate of change of the measure during the time interval between Rₚᵣₑ and Rₚₒₛₜ. A deviation of the trend for each trial from the average trend may also be computed. Each of these values may be collected or computed either as a complex value, or after transformation into separate phase and magnitude components. Therefore, the separate Rₚᵣₑ and Rₚₒₛₜ and R_{trend} measures may be computed for phase and magnitude.

Correction Using Reference Data

The values of the reference deviations may be used to correct the values of the source data for each trial. This may be useful for removing sources of noise that vary trial by trial but are substantially similar or correlated between the source and reference locations. This process may be used to correct either imaging data, single-voxel time course data, multi-voxel time course data, or chemical shift imaging data.

The values of the reference deviations may be used to correct the values of the source data for each trial using the Rₚᵣₑ values by subtracting the Rₚᵣₑ deviation for each trial from the measured source data values S_{1-N} for that trial. This subtraction may be performed using complex data, and/or phase and magnitude data, and/or data transformed to a different coordinate basis. This correction allows fluctuations that affect both source and reference locations to be removed from source data on a trial-by-trial basis. For example, if the starting phase value for the source and reference locations is correlated trial-by-trial due to a noise source, then this correlated noise in the source data may be subtracted out. The reference signal trend (R_{trend}) or trend deviation (R_{trend} deviation) may also be used to separately correct the source signal S deviations over measurements at time points S_{1-N} in a similar fashion by removing the corresponding linear trend from the source data S_{1-N}. R_{trend} deviations may be subtracted from each subsequent value in the series of source data S_{1-N} so that an individual trial's deviation in trend from the average trend is removed from the source data for that trial.

This correction of the source data based upon the reference data may be made through simple subtraction of the start point deviation Rₚᵣₑ, and/or through subtraction of the linear trend deviation R_{trend}. Additional corrections may be used other than subtraction. For instance, if there is a correlation between Rₚᵣₑ values and Sₚᵣₑ or S₁-N values trial-to-trial, then standard statistical methods such as a general linear model may be used to regress out the component of S_{i-N} that can be ascribed to Rₚᵣₑ. If there is a correlation between R_{trend} values and S_{trend} values trial-to-trial, then standard statistical methods such as a general linear model may be used to regress out the component of S_{i-N} that can be ascribed to Rₚᵣₑ and R_{trend}.

Using this correction process, if MR imaging or chemical shift imaging methods are being used, then the values from source k-space data may be reconstructed into image space data after correction of these trial-to-trial variations as described. This method allows correction of image data for short-term fluctuations in magnetic field strength. These may arise from a variety of sources including cardiac cycle, respiration, laboratory noise, magnet fluctuations, data acquisition and demodulation error, and other sources.

Correction Using Source Data

The values of the pre and post source deviations may also be used to correct the values of the source data. In this case, the values of Sₚᵣₑ and Sₚₒₛₜ may be used to correct the values of S_{i-N}. The trial-by-trial deviations of Sₚᵣₑ may be removed from the values of S_{i-N}, or the trial-by-trial deviations of Sₚᵣₑ and S_{trend} may be removed from the values of S_{i-N}. This may be performed using methods similar to those described in the preceding section on correction using reference data.

This correction of the source data based upon the source data may be made through simple subtraction of the start point deviation Sₚᵣₑ, and/or through subtraction of the linear trend deviation S_{trend}. Additional corrections may be used other than subtraction. For instance, if there is a correlation between Sₚᵣₑ values and S_{i-N} values trial-to-trial, then standard statistical methods such as a general linear model may be used to regress out the component of S_{i-N} that can be ascribed to Sₚᵣₑ. If there is a correlation between S_{trend} values and S_{i-N} values trial-to-trial, then standard statistical methods such as a general linear model may be used to regress out the component of S_{i-N} that can be ascribed to Sₚᵣₑ and S_{trend}.

Impedance Measurement or Tomography

The invention disclosed here may be used to measure impedances or impedance changes within objects by correlating changes in electromagnetic fields or currents with corresponding changes in the impedance of the conduction medium, and thereby estimating impedance changes. Tomographic methods may be employed to form 2-D or 3-D maps of impedances or changes in impedance.

Measurements in Electrophysiology

This method may be used to measure the currents and electromagnetic field changes cause by electrophysiological events. In particular, this method may be used to measure the magnitude, location, and direction of current flow within the brain or nervous system that results from electrophysiological activity, whether this activity arises from neurons, glia, other cellular components, or other processes.

Measurements in Contexts Other than Neurophysiology

This method may be used to measure sources of current internal to physical objects. For instance, this method may be used to map the magnitudes, directions and paths of currents flowing within electrical components. In order to accomplish this, an electrical circuit may be placed within the MR measurement apparatus, and differential MR measurements may be made when current is flowing through the circuit, and when current is not flowing through the circuit. This allows measurement of the perturbations in the electromagnetic field surrounding various components of the circuit.

Using the perturbations of the electromagnetic field, it is possible to calculate currents flowing using the Larmour equation and Maxwell's equations. Using the pattern of electric currents and their magnitudes, one may also use this method to make inferences about the components of an electric circuit, for instance, if two current paths originate and terminate at common points and have different currents running through them, then the ratio of the resistances of the two paths can be inferred to be equal to the ratio of the currents, allowing for resistance or impedance measurements. Measurements of the current through a conductor or resistor of known or inferred resistance may also be used to infer the voltage across the conductor or resistor. Measurements of the time rate of change of current leading into a capacitative component may also be used to infer capacitance. Similar logic may be useful to infer other properties of an electric circuit, such as inductance, the state of switches, the state of logic circuits, and operations taking place within integrated circuits. This method may also be used to measure MR eddie currents.

Measurements of Other Physiological Processes

This method may be used to measure currents generated by processes outside of the brain, such as magnetic field perturbations or currents arising from spinal cord, peripheral or cranial nerves, muscles and cardiac tissue. In the case of the measurement of peripheral nerve, muscle, and spinal cord, the measurement principles are substantially similar to those for measurement of brain neurophysiologic processes. The perturbation in magnetic field associated with the activation of a peripheral nerve may be measured by comparing MR signals in the presence and absence of a stimulus that may activate the nerve. Such stimuli may include direct electrical or magnetic stimulation of the nerve, sensory stimulation of the receptors enervating the nerve, or movements carried out through activation of the nerve. The perturbation in magnetic field associated with the activation of muscle tissue may be measured by comparing MR signals in the presence and absence of a stimulus that may activate the muscle. Such stimuli may include direct electrical or magnetic stimulation of the muscle, or movements carried out through activation of the muscle.

The perturbation in magnetic field associated with the activation of cardiac tissue may be measured by comparing average MR signals at different points in the cardiac cycle. This may be accomplished through cardiac gating, leading to the measurement of MR signals that take place at different times relative to the onset of a cardiac cycle. In addition, through the measurement of a timecourse of MR data over the course of part of a cardiac cycle, the timecourse of currents associated with the cardiac cycle may be measured.

Resting State and EEG Rhythm-Type Activity

The information derived using this method may be used to estimate resting state brain activation and EEG rhythm-type activity. The data obtained using this invention from a source location may be used to compute the power spectrum of neurophysiological activity arising from that location. This power spectrum may be used to determine the dominant frequencies of activation. The data obtained using this invention from a source location may be used as input to band-pass filters to determine the level of activity in different frequency bands. Power spectrum and frequency band information may be used from one or more brain location to determine the level of brain rhythmic activity, such as alpha, beta, delta and gamma activity previously measured using EEG. This invention may be used to localize the current generators of EEG-measured currents and other neurophysiological currents.

Combination with Other Methods

The methods described here may be made in combination with a variety of other methods. For example, the measures described here, which may be designated as emfMRI measures in some contexts, may be compared with or correlated with other measures arising from physiological measurement means that include, but are not limited to: functional magnetic resonance imaging (fMRI), BOLD imaging, PET, SPECT, EEG (electroencephalogram) recordings or event-related electrical potentials, MEG recordings (magnetoencephalogram), electrode-based electrophysiological recording methods including single-unit, multi-unit, field potential or evoked potential recording, infrared or ultrasound based imaging methods, or other means of measuring physiological states and processes. In addition, this method may be used in combination with stimulation methods such as electrical stimulus, or transcutaneous magnetic stimulation to determine the perturbations in neurophysiological activity caused by these stimulation methods.

This method may also be used in combination with pharmacological methods to determine the perturbations in neurophysiological activity caused by pharmacologic agents, in the presence or absence of additional stimulation methods. This method may be used in combination with pharmacologic testing. This method may be used to derive information that may be processed as described in the section Use in combination with pharmacologic testing of US Appl. Publ. No. 20020103429.

Information about electromagnetic fields arising from neurophysiological events may be used in additional contexts. This information may be used as a physiological measurement for all of the methods described in US application 20020103429 and provisional application 60/399,055 “Methods for Measurement and Analysis of Brain Activity”. Specific examples including using the information derived from this invention as measured of physiological activation for use as described in the following sections of that application: Localization of neuronal function, especially for neurosurgery, Localization of seizure foci, Diagnosis and treatment of neurologic injury, Mapping and diagnosis of areas of injury or disease, Treatment of areas of injury or disease, Characterization of brain regions.

Contrast Agents

It is noted that contrast agents may be optionally used in combination with the methods described here for physiological signal measurement when performing the various methods of the present invention. By using contrast agents to assist brain scanning, it may be possible to achieve larger and more reliable activation measurements. Examples of exogenous contrast agents that may be used in conjunction with the methods of the present invention include, but are not limited to the contrast agents disclosed in U.S. Pat. No. 6,321,105.

Measurement of Neuronal Activity using Additional Means

This invention may be used in conjunction with a variety of means for measuring physiological activity from a subject. Examples of measurement technologies include, but are not limited to, functional magnetic resonance imaging (fMRI), PET, SPECT, magnetic resonance angiography (MRA), diffusion tensor imaging (DTI), trans-cranial ultrasound, trans-cranial doppler shift ultrasound, infrared spectroscopy (NIRS), BOSS fMRI imaging, cardiac monitoring (ECG), pulsoximetry, respiratory monitoring, electrophysiological measures including EEG, EMG, nerve conduction measurement, peripheral nerve stimulation. It is anticipated that future technologies may be developed that also allow for the measurement of activity from localized brain regions, preferably in substantially real time. Once developed, these technologies may also be used with the current invention. These measurement techniques may also be used in combination, and in combination with other measurement techniques such as EEG, EKG, single neuronal recording, local field potential recording, ultrasound, oximetry, peripheral pulsoximetry, near infrared spectroscopy, blood pressure recording, impedance measurements, measurements of central or peripheral reflexes, measurements of blood gases or chemical composition, measurements of temperature, measurements of emitted radiation, measurements of absorbed radiation, spectrophotometric measurements, measurements of central and peripheral reflexes, and anatomical methods including X-Ray/CT, ultrasound and others.

Any localized region within the brain, nervous system, or other parts of the body that is measured using physiological monitoring equipment as described (or other physiological monitoring equipment that may be devised) may be used as the region of interest of this method. For example, if measurement equipment is used for the monitoring of activity in a portion of the peripheral nervous system, such as a peripheral ganglion, then subjects may be trained in the regulation of activity of that peripheral ganglion. In addition, this invention may be used to monitor the perturbations of magnetic field associated with the vasculature of the brain, and with other bodily areas, which may serve as regions of interest.

Combination with rtfMRI Training Methods

The methods described herein may be used in the training of subjects to control brain activation, as described in U.S. Patent Application 20020103429 “Methods for physiological monitoring, training, exercise and regulation”. Specifically, measures of the perturbation of a magnetic field derived here may be considered as an indication of neuronal activation. This indication of neuronal activation may be used as a functional magnetic resonance imaging (fMRI) measure. This FMRI measure m ay be used to train subjects to control brain activation in the target region of interest as provided for by methods described in U.S. Patent Application 20020103429.

Programmable Computer and Software

Any of the methods described herein may be performed using a programmable computer. Such a computer can include a central processing unit connected to a set of input/output devices via a system bus. The input/output devices may include a keyboard, mouse, scanner, data port, video monitor, liquid crystal display, printer, and the like. A memory in the form of a primary and/or secondary memory may also be connected to the system bus. These, and other components that may be included, are characteristic of a standard computer. Such a computer is preferably programmable. In particular, the computer can be programmed to perform various operation of the methods of the present invention, for example, receiving MR signals, amplifying MR signals, producing free induction decay, differentially measuring free induction decay, comparing data from the processes herein from data derived from other physiological measurements.

In some embodiments, the memory of the computer stores test and reference MR signals. The memory may also store a comparison module. The comparison module includes a set of executable instructions that operate in connection with the central processing unit to compare various MR signals, free induction decay patterns, phase and magnitude data, etc. The executable code of the comparison module may utilize any number of numerical techniques to perform comparisons.

The memory also stores a decision module. The decision module includes a set of executable instructions to process data created by the comparison module. The executable code of the decision module may be incorporated into the executable code of the comparison module. In preferred embodiments, the decision module includes executable instructions to provide a decision regarding the presence or absence of a significant MR differential measurement.

### EXAMPLES

Theoretical Basis and Previous Investigations of MR Phase Measurement

Precise measurements of Bo fluctuations using MR are explained by the relation that σφ=1/SNR, whereσφ is the MR phase noise in radians, and SNR is the signal to noise ratio of the MR magnitude signal. The phase value may be substituted into the Larmour equation (expressed in terms of phase): Δφ(r)=γBz(r)TE, where Δφ(r) is the change in phase at a point r resulting from a perturbation of the Bz, TE is the duration of phase accumulation prior to measurement, and γ is the magnetogyric ratio. At 1.5T, an MR signal resonates over 6.4 million cycles during a 100 ms period. Since the MR phase signal represents a small fraction of one cycle, a modest phase precision of 1/100^{th} of a cycle (0.06 radians) at 100 ms predicts a ΔB₀ measurement precision of 1 part in 100×6.4 million, or 4×10⁻⁹ T. Therefore, MR phase measures B₀ fluctuations with surprising precision. Nyquist sampling theory limits the frequency resolution (linewidth) of MR measurements to much poorer resolution than suggested here, based upon the sampling bandwidth, because a relatively broad bandwidth MR signal is typically acquired and then Fourier transformed to achieve frequency separation. Here, much higher resolution is possible because small phase accumulations over time are measured relative to a very narrow-band carrier frequency.

Measurements of Neuronal Currents May be Limited by Physiology—Comparison with Electrophysiology

The direct measurement of neuronal currents in vivo is primarily a challenge of overcoming physiological noise. Therefore, known principles from neurophysiology may be used to solve this problem. In order to make satisfactory measurements of neuronal currents, it is possible to use differential measurements that allow for common-mode noise rejection. It is also possible to record high-frequency time series data and then employ band-pass filtering or subtraction. Together, these techniques may substantially decrease noise in neurophysiology, and are likely similarly applicable to measurements using MR. Differential recording principles may be applied by making measurements from two receive coils at high temporal sampling rate, using differential processing of the two data streams (rather than a linear combination typically used in MR multi-coil or phased-array acquisition), and removing high-frequency noise and lower frequency physiological fluctuations through filtering or subtractive methods of time-course MR phase data.

Differential measurement of MR phase as described here may require sampling time series MR phase data from multiple receive coils—the subtraction of values from different spatial voxels obtained using an imaging sequence may not accomplish the same result. When using a single coil in conventional imaging, phase values from two different voxel locations in image-space are derived by Fourier transform from k-space data collected over the same readout period for both voxels. Therefore, shifts in B₀ that take place on a physiological time scale may not be corrected for accurately by voxel-wise subtraction. Since spatial information in MR imaging is encoded in phase, changes in the underlying magnetization phase during readout are interpreted as spatial information rather than changes in resonance frequency.

Measurement of MR Phase Timecourse with Millisecond Precision

Previous measures of MR phase have used MR imaging, which typically generates a single complex value for each voxel following each RF excitation. (TR). The methods proposed here acquire an entire free induction decay (FID) from a voxel using spectroscopic techniques, and thereby allow reconstruction of the entire MR phase timecourse from the voxel over several hundred milliseconds following an RF pulse as shown in FIG. 2. On each trial, stimulus presentation may be precisely synchronized to the time of RF excitation. The stimulus time is adjusted so that the evoked neuronal response falls during the FID. The FID may be recorded at millisecond temporal resolution or better, and is converted into a timecourse of MR phase. The change of this MR phase timecourse reflects the change in B₀ field associated with the measured EMF signal. The MR phase timecourse is then compared for trials with and without a stimulus, and band-pass filtering or temporal difference measures may be applied to further reduce physiological noise.

Methods for Data Acquisition and Analysis to Reduce Phase Noise

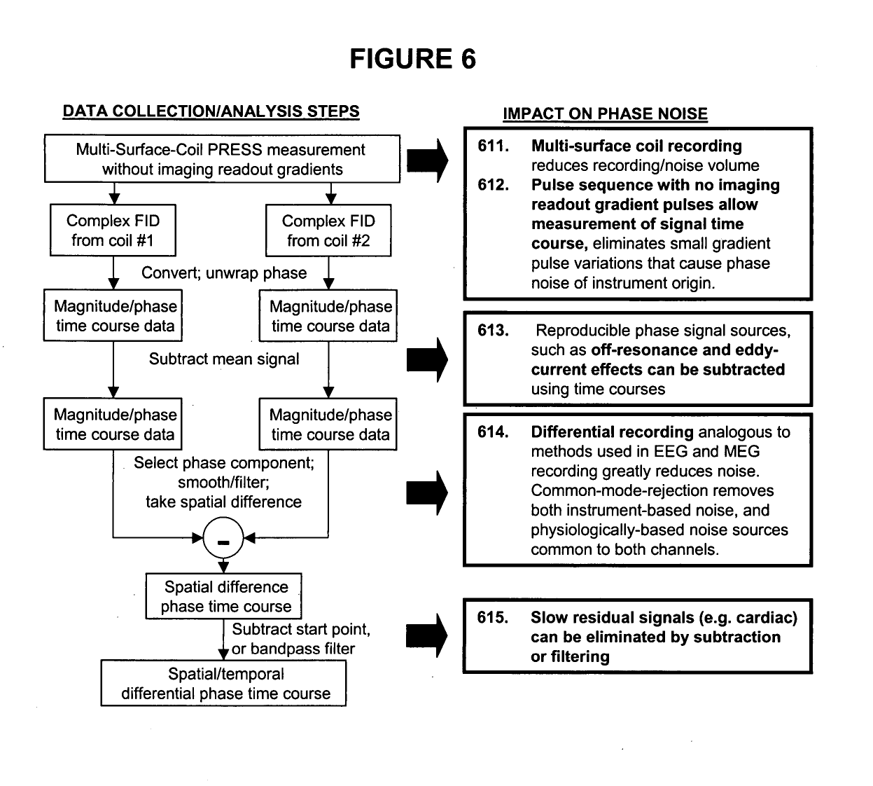

Disclosed is a combination of five significant innovations not previously applied to the problem of the measurement of currents using MR (outlined in FIG. 6). These five improvements are based upon the novel approach of using continuous FID measurements from spectroscopic techniques, rather than MR imaging measurements used in the past. They include:

Multi-Coil MR Recording of Electromagnetic Field Perturbations Using Surface Coils 611

In order to decrease the MR noise volume, a custom-built multi-coil system employing surface coils adjacent to the area being measured may be used. Surface coils have sufficient coverage to record deep brain structures, as well as visual cortex and optic nerve. These methods may be adapted for volume measurement with phased-arrays or volume head coil/surface coil configurations.

Using Spectroscopic Pulse Sequences without Imaging Gradients to Measure Electromagnetic Field Perturbations 612

Conventional MR imaging methods use a sequence of gradient pulses during data readout to allow k-space localization for subsequent spatial reconstruction. Since imaging requires a sequence of multiple gradient pulses during readout, any small variability of these successive gradient pulses leads to cumulative total phase error. The MR spectroscopy sequence utilized here may only use gradient pulses during the excitation phase, not during the readout phase, leading to greater phase stability. This PRESS sequence [Bottomley (1987). “Spatial localization in NMR spectroscopy in vivo.” Ann N Y Acad Sci 508: 333-48.], achieves spatial localization using a slice-selective excitation pulse followed by two slice-selective refocusing pulses, each along a different axis. This produces signal only from a rectangular voxel without the need for any additional gradients for localization. This is a distinction from imaging sequences that achieve spatial localization by applying gradients during the readout period. By using a low-noise, single-voxel technique adapted from spectroscopy, it is possible to eliminate many sources of system instability, such as gradient heating, gradient amplifier loading, vibrational motion, as well as greatly reducing eddy-current induced phase shifts.

Collection of MR Phase Timecourse Data and Subtraction of Average Timecourse to Measure Electromagnetic Field Perturbations 613

Imaging methods typically provide only a single complex value for each voxel following each RF excitation/acquisition, not a timecourse. The PRESS spectroscopy pulse sequence uses no gradient pulses during readout, so it allows measurement of the timecourse of the MR phase signal as it evolves in time over a period comparable to an evoked-potential response (several hundred ms, limited by the T2*). This allows the timecourse of neuronal current to be directly explored. Timecourse information may be used either to probe stimulus-evoked responses, or spontaneous activity (e.g. spontaneous alpha).

The measurement of a full phase timecourse instead of a single time point has important implications for noise removal. The phase signal in time is affected by multiple sources such as off-resonance, eddy-currents, external magnetic field fluctuations, and B₀ instabilities. In order to remove components that are common from shot to shot, the average phase timecourse is subtracted from the phase timecourse observed following each individual acquisition period. This removes large common components, and leaves only the residual phase timecourse, which may be sensitive to changes in phase signal that differ from readout to readout (such as stimulus-evoked components).

This may be performed using single-voxel methods. PRESS may also be combined with phase encoding (PRESS-CSI) prior to acquisition to achieve 2D and 3D spectroscopic imaging. PRESS-CSI may be used for collection of MR Phase Timecourse Data from a spatial array of locations with subsequent Subtraction of Average Timecourse to Measure Electromagnetic Field Perturbations. This allows the acquisition of a full FID, and resulting MR phase timecourse, from each voxel in a grid or volume. Due to the k-space nature of the signal, full timecourse data may be acquired for an array of voxels with little or no penalty in acquisition time or SNR compared to the collection time of a single voxel of equivalent size [Star-Lack, Vigneron et al. (1997). “Improved solvent suppression and increased spatial excitation bandwidths for three-dimensional PRESS CSI using phase-compensating spectral/spatial spin-echo pulses.” J Magn Reson Imaging 7 (4): 745-57; Lin, Fertikh et al. (2000). “2D CSI proton MR spectroscopy of human spinal vertebra: feasibility studies.” J Magn Reson Imaging 11 (3): 287-93.].

Differential Recording, and Common-Mode Noise Rejection to Measure Electromagnetic Field Perturbations 614

It is common practice in electrophysiological measurements, such as EEG, MEG, and single neuron recording, to simultaneously measure a target signal and a nearby reference signal, and observe the difference between the two. Differential recording eliminates noise common between two recorded locations, such as background noise, and cardiac and respiratory signals that would otherwise dwarf the signal to be measured. Standard neurophysiological measurements of neuronal current would be impossible due to noise without differential recording, just as measurements of neuronal current with MR has been impossible to date. If voxels are placed a similar distance from cardiac and respiratory noise sources, they receive similar noise [Menon (2002). “Postacquisition suppression of large-vessel BOLD signals in high-resolution fMRI.” Magn Reson Med 47 (1): 1-9.].

In order to achieve differential recording using MR, two or more separate receive coils may be employed. By comparing the timecourse of the phase signal derived from two separate coils that have separate areas of spatial coverage (which can be precisely shaped during excitation using saturation bands), it is possible to perform true differential measurement. The phase and/or magnitude timecourse from each of two receive coils is subtracted to yield differential recordings. In order to further improve this process, it is also possible to use a linear model to separately fit the data from the two coils for each readout sample point in time, and use the residuals from this model as the differential signal, yielding a further improvement in some cases.

Temporal Filtering/Difference Measures to Measure Electromagnetic Field Perturbations 615

Finally, and also in analogy to electrophysiology, residual fluctuations outside the desired neuronal frequency band may be removed by either band-pass filtering, or subtracting early time points in the trace from later time points to achieve a temporal difference. The millisecond-level temporal filtering of MR phase described here (e.g. bandpass 5-100 milliseconds⁻¹) is entirely distinct from the filtering of slow BOLD magnitude signals typically used in fMRI (e.g. bandpass 5-60 seconds⁻¹).

MR Scanner and Equipment

The methods outlined may be performed using a variety of measurement instruments. Examples of measurement are next presented. Scanning may be performed using a 1.5T GE Medical Systems Signa LX MRI system equipped with high performance gradients (40 mT/m, 150 T/m/s slew rates). MR measurements may be performed using a custom designed and built dual-surface-coil head imaging system. This system incorporates a form-fitting, rigid motion restraint system that precisely positions the surface coils relative to the subject's head, and minimizes head motion.

Current Phantom

A phantom without metal conductors has been built to allow the testing of injection of current. The phantom is a vessel containing dilute CuSO₄ solution, with 2 mm plastic tubing running through it containing conductive CuSO₄/saline. Current may be injected using electrodes well outside of the receive area of the coils, and is conducted through the tubing. The resistance through the tubing is approximately 20 kΩ, and additional resistance can be applied through in-line resistors to adjust the current supplied using a constant 9V source.

### EXAMPLE

### Pulse Sequences

MR Phase Timecourse Measurement

Electrical current measurements may be made using a single-voxel PRESS technique that is part of the standard GE product spectroscopy package and is in routine use in MR spectroscopy. This pulse sequence decreases phase noise during electrical current measurement compared with imaging methods by eliminating gradient pulses during data readout. Rather than exciting the entire volume and then using imaging gradients during readout to achieve spatial specificity, this sequence initially excites only a spatially defined 3D region, and then performs readout from this region with no additional gradients applied. Phase noise caused by small gradient inconsistencies may thus be eliminated. The PRESS sequence selects a 3D voxel using a 90-180-180 sequence of RF pulses, each along one of the x, y, and z axes. The MR signal is produced only by the voxel defined by the intersection of these three slice-selective pulses. Additional shaping of the voxel may be performed using GE's Very Selective Saturation (VSS) pulses [Le Roux, Gilles et al. (1998). “Optimized outer volume suppression for single-shot fast spin-echo cardiac imaging.” J Magn Reson Imaging 8 (5): 1022-32.], which may also be used to suppress designated regions of the excited voxel.

Selection of Target and Reference Voxels For Differential Measurement

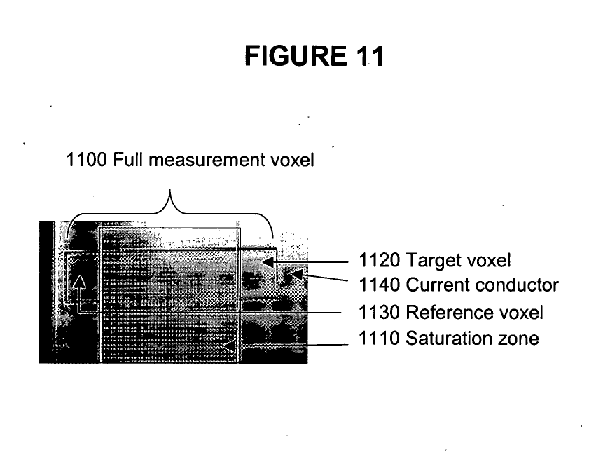

To allow differential measurement, two excitation voxels may be selected based upon an initial T1 localization scan. One voxel may be adjacent to each of two receive coils. The prescription image in FIG. 11, captured during a preliminary experiment, shows an example from the conducting phantom. A large rectangular measurement voxel 1100 is first selected. A saturation zone 1110 is then applied to remove signal from the central region of this large rectangle, separating it into a target voxel 1120 (right) and a reference voxel 1130 (left). In this case, the target voxel is adjacent to a current conductor 1140 (which runs through plane), and also adjacent to the sensitive volume of a 5″ receive coil (corresponding to the high signal intensity region, top). The reference voxel is adjacent to the receive area of a 3″ receive coil (left).

### EXAMPLE

### Stimulus Presentation and Synchronization

Synchronization to Scanner

Sensory stimuli or current pulses may be precisely synchronized with the measurement pulse sequence using a dedicated synchronization computer system with a high-speed analog to digital converter that serves as a trigger-detector. The stimulus is initiated after a software controllable delay. For phantom measurements, the synchronization computer puts out analog or digital waveforms that modulate or gate DC current driven through a resistive circuit. DC current pulses are presented using custom circuitry that isolates DC currents injected into the phantom from AC currents generated by lab equipment or induced by RF or gradient pulses within the scanner, and includes appropriate low-pass RC components to reduce any MHz-frequency pickup. The current is monitored by oscilloscope during scanning to ensure a clean waveform and correct time synchronization. Current amplitude is calibrated by measuring resistance and voltage drop through the conductive tubing within the phantom itself.

Visual Stimulus Presentation and Perceptual Control

Visual stimuli may be back-projected onto a translucent screen viewed in an angled mirror by subjects while within the scanner bore, using a DLP video projector. Stimuli may consist of 50 ms long flashed presentations of a high spatial frequency, high contrast checkerboard annulus. Subjects may be instructed to fixate continuously on the screen center. The precise timing of stimuli relative to pulse-sequence presentation may be monitored during experiments using a photodiode to measure the light intensity of the stimuli, whose output may be displayed on an oscilloscope. Stimuli may also be presented using a strobe that can be triggered relative to the pulse-sequence time, which back-illuminates a pattern. Subject attention may be directed toward the stimulus and continuously monitored by instructing subjects to indicate using a response device whenever they perceive a slightly colored version of the stimulus.

Stimulus Sequencing

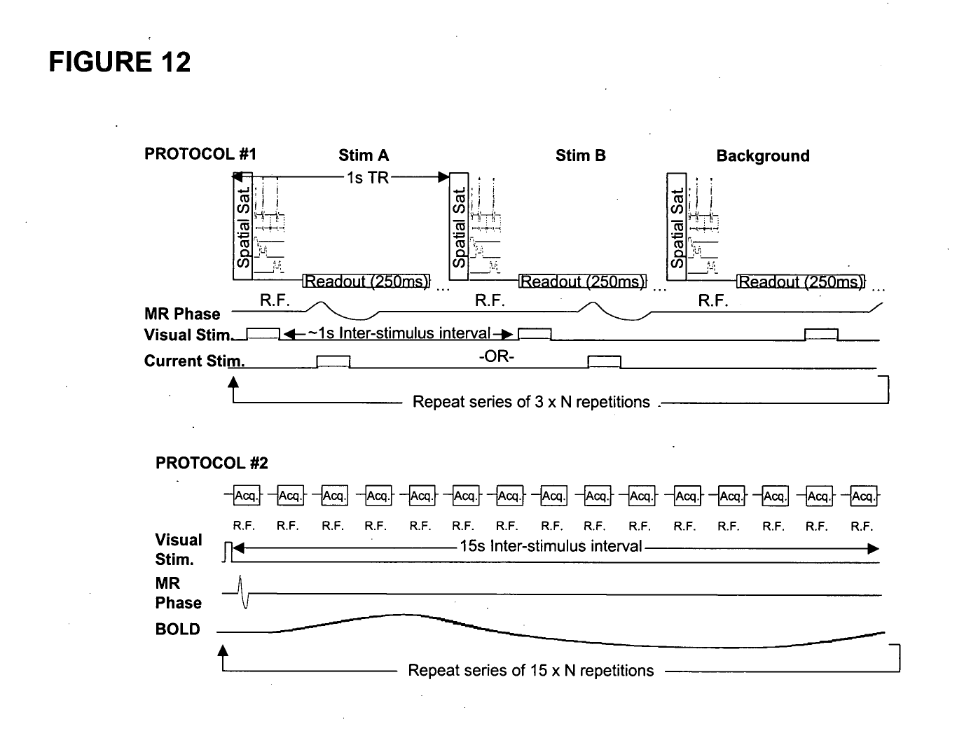

Stimuli may be sequenced as depicted in FIG. 12 protocol #1. Individual acquisitions may be separated by a period (TR) of is. Stimulation may follow a repeating cycle of three conditions. Visual stimuli or currents may be presented at times synchronized to the acquisitions so that in the Stim A condition the start of the evoked or injected current coincides with a time slightly after the start of the readout period. In the Stim B condition the neural or injected current arrives later in the readout period. In the background condition, there is no current during the readout period. In the background condition, a visual stimulus is still presented, but is arranged so that the evoked neural response comes after the readout period has concluded. This ensures that the three conditions are nearly identical as relates to visual perception, and processes operating on a slower timescale (e.g. BOLD).

### EXAMPLE

### Optimization and Characterization of Methods Using a Current-Conducting Phantom

Rationale Results suggest that it is possible to measure the minute Bo fluctuations expected to accompany neuronal activation. In order to optimize measurement parameters, and fully characterize the method, MR phase measurements may be made in a simple current-conducting phantom.

Protocol Single coil and differential MR magnitude and phase may will be measured from a target voxel adjacent to a current-conductor in a phantom, and a reference voxel located away from any current source. Current injection may be sequenced as described above under Stimulus Sequencing, and the MR phase and magnitude timecourses may be compared between trials when current was or was not injected. The following parameters may be parametrically adjusted: voxel position, voxel size, TR, TE.

Measures and Analyses MR data may be measured and processed as described above in Methods for Data Acquisition and Analysis. The standard error of the timecourse of the phase signal on successive RF shots may be measured. In addition, the phase timecourse may be compared for successive trials with and without injected current in order to determine the smallest B₀ fluctuation that may be detected.

Results and Discussion

1) Measurement precision may be significantly improved using differential recording methods.

2) Feasibility may be demonstrated by showing measurement accuracy sufficient to measure estimated fields induced by neuronal currents in vivo (100pT), using current appropriate to generate the requisite signal (10-100 uA).

3) Optimal conditions may include minimum TE, TR of ˜0.5-1.5s, voxel side of ˜0.5-2 cm³.

4) Phase signals may reverse on opposite sides of the injected current, with the phase signal maximal parallel and anti-parallel to the main B₀ field.

### EXAMPLE

### Measurement of Fast Neuronal Signals in the Human Brain, Separation from BOLD Signals

Rationale In order to induce a repeatable neuronal current, a highly salient, flashed visual stimulus may be presented to subjects using a DLP projector or strobe. To distinguish rapid neuronal signals from slower signals associated with hemodynamic effects such as BOLD, in a second experiment MR measurements may be made at delays of 0-15s following stimulus presentation. The current-related MR signal may reach its maximum within tens to hundreds of milliseconds from the onset of a visual stimulus, and may be substantially attenuated or absent when the BOLD signal reaches its maximum value at ˜5s post-stimulus.

Protocol In order to physiologically target measures, activated regions of the visual cortex may be localized using substantially real time BOLD fMRI [Cox, Jesmanowicz et al. (1995). “Real-time functional magnetic resonance imaging.” Magn Reson Med 33 (2): 230-6; Voyvodic (1999). “Real-time FMRI paradigm control, physiology, and behavior combined with near real-time statistical analysis.” Neuroimage 10 (2): 91-106; Gembris, Taylor et al. (2000): “Functional magnetic resonance imaging in real time (FIRE): sliding-window correlation analysis and reference-vector optimization.” Magn Reson Med 43 (2): 259-68; Posse, Binkofski et al. (2001). “A new approach to measure single-event related brain activity using real-time fMRI: feasibility of sensory, motor, and higher cognitive tasks.” Hum Brain Mapp 12 (1): 25-41; Yoo and Jolesz (2002). “Functional MRI for neurofeedback: feasibility study on a hand motor task.” Neuroreport 13 (11): 1377-81.]. This localization may be performed using a block design of 15s of a 10 Hz reversing annulus grating followed by 15s of a blank screen. Conventional substantially real time FMRI data may be used to select a voxel location maximally activated by a visual stimulus for further investigation.

Differential MR phase data may then be measured using a target voxel selected in either visual cortex or adjacent to optic nerve with a 5″ surface coil adjacent to the target voxel. A reference signal may be measured from a second voxel in a frontal or temporal region at a similar distance from the chest using a second receive coil. Stimuli may be presented as described in FIG. 12, Protocol #1. In FIG. 12, Protocol #2, only a single flashed visual stimulus may be presented at the beginning of each 15s interval, but MR phase measurements may be collected for 250 ms epochs at one second increments throughout the interval, up to a maximum increment of 15s before the next stimulus is presented. The methods proposed here do not allow continuous high-frequency measurement of MR phase (due to T2*), but can measure 250 ms blocks of data at 1s intervals.

Measures and Analyses MR data may be measured and processed as described in Methods for Data Acquisition and Analysis. The timecourse of the MR phase and magnitude may be measured over a 250 ms period following each RF excitation. The MR phase may be compared for periods following a stimulus and for periods when a stimulus was not presented, in both protocol #1, and protocol #2. In addition, the MR magnitude from each RF shot (with TE appropriate to BOLD measurement) may be simultaneously acquired from each readout period to allow simultaneous measurement of the BOLD effect.

MR phase timecourse +/− standard error may be computed to determine whether statistically significant differences in phase can be detected when comparing conditions with a stimulus from background conditions. A single time point may be selected for all subjects as the point of maximum stimulus-evoked phase response after the RF excitation. The phase at this time point may be compared between stimulus and background conditions for each subject (paired t-test), and also across the subject group (one way ANOVA with repeated measures). In addition, the amplitude of the MR phase at this time point may be compared for all 15 acquisitions in protocol #2 in order to determine whether the phase response from the first RF excitation is greater than for succeeding RF excitations. The timecourse of the fMRI BOLD hemodynamic response may also be computed as the magnitude of the MR response at each of the 15 time points following the stimulus.

Results and Discussion

1. MR phase shifts associated with neuronal activation may be reproducibly measured using this method.

2. These phase shifts follow a rapid timecourse after the presentation of a stimulus.

3. MR phase shifts associated with stimulation far precede the onset of the BOLD effect.

4. There may be larger MR phase shifts measured from optic nerve, a linear structure, than from cortex, due to the alignment of the neuronally-induced currents.

### EXAMPLE

### Measurement of Phase Timecourse in Current Phantom

Rationale MR phase is a sensitive indicator of small fluctuations in the B₀ field. The methods tested here lead to highly precise measurements of MR phase in vitro.

Methods The MR phase timecourse may be measured from a current-conducting phantom. Briefly, a PRESS spectroscopy pulse sequence may be used to measure the MR phase timecourse from two voxels—one voxel in the receive area of each of two receive coils. The phase timecourse data from these two coils may then be subtracted, and the mean of resultant differential phase timecourses may then be further subtracted from each individual differential phase timecourse. Finally, the mean remaining value of each acquisition trace may be subtracted. This corrects for spatial noise, average eddie current noise, and residual slow fluctuations. The target voxel may be adjacent to a current conducting tube of saline within the phantom, while the reference voxel may be distant.

Protocol On repeated trials, either no electrical current may be injected through the phantom, or current may be injected during the MR acquisition.

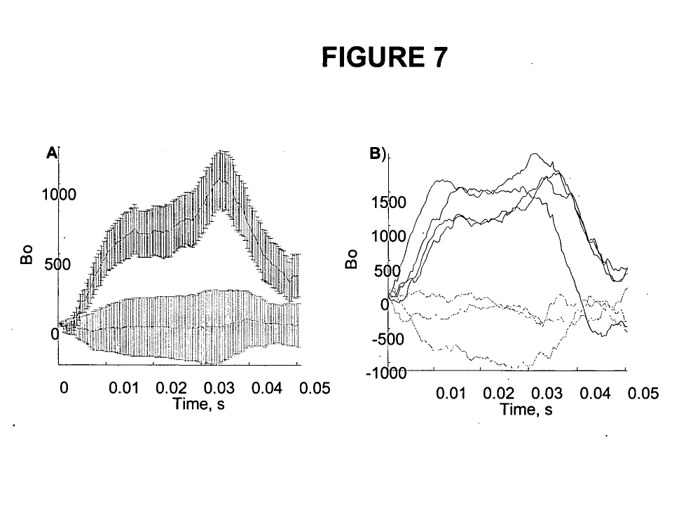

Results Experiments verify that it is possible to measure MR phase with an accuracy (assessed as standard error) better than 100pT. FIG. 7 shows the average trace for each of two conditions, one with a current pulse applied, and the other with no current applied. The standard errors of 15 measurements of each condition, shown as error bars, indicate that even with little averaging, impressive precision is possible. FIG. 7B shows example traces of cases with and without applied current. The applied current was a ˜40 ms stimulus, corresponding to a predicted B₀ fluctuation of 660pT at the recording distance (15 mm voxel center to current source).

Relevance and Discussion Extremely precise measurements of small B₀ fluctuations are possible using this method, even with little signal averaging. Neuronal currents measured close to their generators inside the brain are in the measurable range (hundreds of picoTesla).

### Example

### Differential Recording of MR Phase In Vivo

Rationale MR phase in vivo is substantially perturbed by physiological noise sources. Using differential methods, it is possible to remove a substantial portion of this noise.

Methods and Protocol A target voxel may be placed in the occipital cortex of a subject, adjacent to a 5″ receive coil, and a reference voxel may be placed in frontal lobe, at approximately equivalent distance from the chest, adjacent to a second receive coil. No stimuli are presented in this example. Methods were as in preliminary study 1.

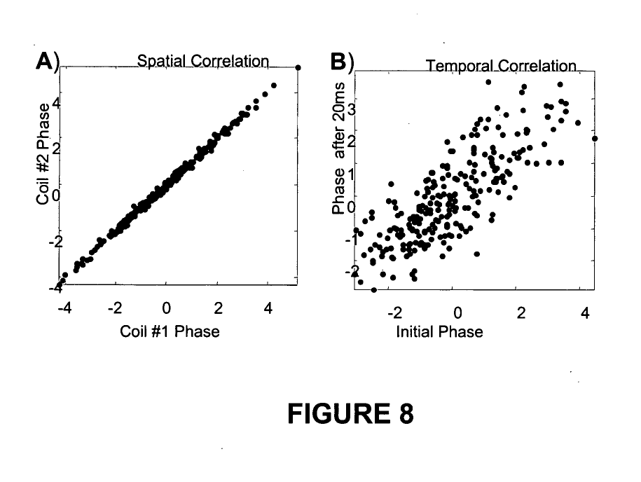

Results FIG. 8A shows the correlation in MR phase following individual RF shots between the signal collected from two voxels measured by separate receive coils. In the thermal-noise limit, these two signals would be uncorrelated. However, substantial correlation is observe (r=0.988). This common-mode noise may be removed by subtracting the signal from the two voxels to achieve differential recording. This noise is presumed to be primarily contributed by cardiac and respiratory processes, and environmental noise sources.

After computing the difference in phase between the two coils, the residual temporal correlation is decreased. FIG. 8B shows the remaining correlation in MR phase following individual RF shots between the initial phase measure, and a second measure taken after 20 ms. This temporal correlation (r=0.965) suggests that the mean time point may be subtracted from remaining time points to remove slow variations in the MR phase timecourse, or the timecourse may be bandpass filtered from 10-100 Hz (thereby also removing the contribution of trace mean).

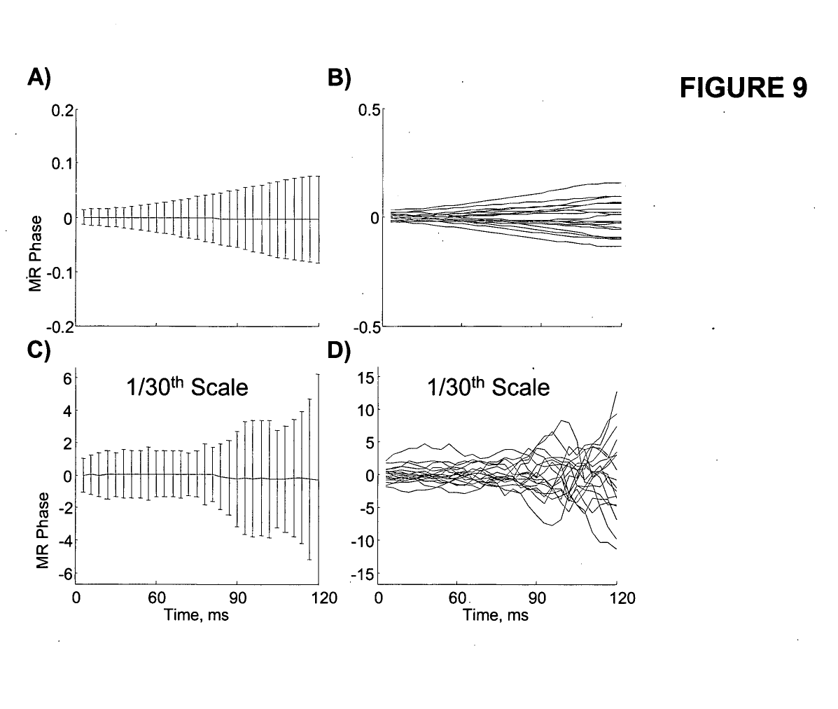

FIG. 9 demonstrates the improvement in the in vivo phase timecourses that may be achieved using spatial and temporal difference measures. FIG. 9A represents the mean phase signal from a single coil, with the standard deviation of measurement shown. FIG. 9B shows example individual traces. FIGS. 9C and 9D show the standard deviation and example traces from the phase timecourse computed by subtracting the two coils, and subtracting each trace's mean. An improvement in phase noise by a factor of greater than 30:1 is achieved in this example, with the improvement greatest at about 75 ms.

Relevance and Discussion Scanner phase stability is adequate for the purposes of conventional MR imaging, although there have recently been efforts to decrease physiological phase noise for high field measurements [Pfeuffer, Van de Moortele et al. (2002). “Correction of physiologically induced global off-resonance effects in dynamic echo-planar and spiral functional imaging.” Magn Reson Med 47 (2): 344-53.]. However, for the purpose of measuring very small phase perturbations associated with neuronal current, differential measurement methods are useful. In the data presented, an ˜30-fold improvement in phase noise was observed.

### EXAMPLE

### Measurement of Neuronal Currents In Vivo

Rationale Neuronal currents lead to fluctuations in B₀ with magnitudes within the precision of methods disclosed here. Therefore, the MR phase timecourse of FIDs may be compared with and without an evoked neuronal response to determine the effect of the neuronal response. In order to induce a repeatable neuronal current, a highly salient, flashed visual checkerboard stimulus may be presented to subjects, precisely synchronized to MR measurements lasting for 120 ms following the stimulus.

Protocol Differential MR phase data may be collected using a target voxel selected in the visual cortex with a 5″ surface coil placed adjacent, while a differential signal was measured from a second voxel in a frontal region using a second receive coil a similar distance from the chest. Stimuli may be presented using a DLP projector as diagrammed in Visual Stimulus Protocol #1.

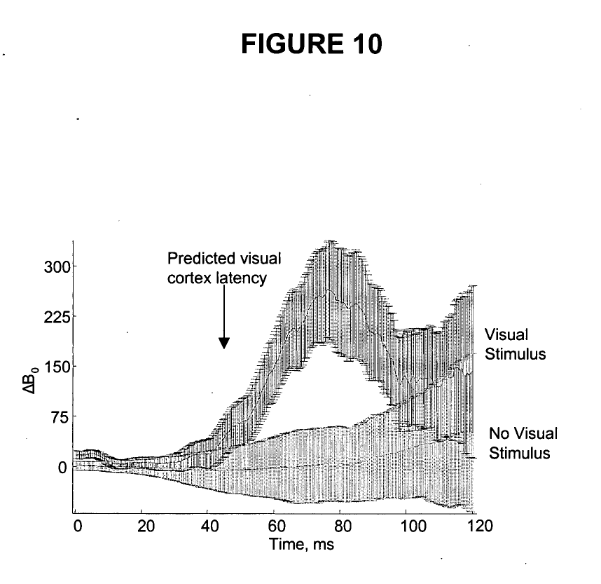

Results Neuronal currents may be measured using this approach. The example presented in FIG. 10 depicts the mean of MR phase signal from trials collected with and without the presentation of a visual stimulus, with standard error of the mean of 40 repetitions shown. This measurement corresponds to a peak B₀ fluctuation with onset latency of ˜48 ms.

Relevance and Discussion These data demonstrate the measurement of neuronal currents in vivo using MR. The signal shown has a magnitude in the range predicted by models, and a latency as predicted by known visual cortex MEG/EEG signal latencies. The data presented used only 40 presentations of each condition. Greater response averaging may likely lead to improved measurement reliability. Further investigations may use substantially real time FIMRI to target voxels to maximally activated brain regions. Finally, the presented measurements were carried out in visual cortex. It is also possible that measurements from optic nerve or optic tract may show greater B₀ fluctuations due to higher current densities found within an oriented nerve bundle.

It will be apparent to those skilled in the art that various modifications and variations can be made to the methods, software and systems of the present invention. The foregoing examples and figures are presented for purposes of illustration and description. It is not intended to be exhaustive or to limit the invention to the precise forms disclosed. Many modifications and variations will be apparent to practitioners skilled in this art and are intended to fall within the scope of the invention.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

1. A device to measure neuronal currents comprising:
a means for reference MR signal amplification;
a means for test MR signal amplification;
and a means for determining the difference between the reference MR signal and the test MR signal.

2. The device of claim 1 wherein the reference MR signal and the test MR signal are measured simultaneously.

3. The device of claim 1 wherein the neuronal currents are induced by a neural activation.

4. The device of claim 3 wherein the neuronal activation is selected from the group consisting of a visual image, a visual sequence, an auditory sound, an auditory sequence, a tactile sensation, an electrical stimulus to a peripheral location, an electrical stimulus to the central or peripheral nervous system, a pharmacological or other physiological stimulus, a perceptual stimuli, an instruction, and a set of instructions.

5. The device of claim 1 further comprising means for determining free induction decay of the amplified reference MR signal and amplified test MR signal.

6. The device of claim 1 further comprising means for determining free induction decay of the amplified reference MR signal and amplified test MR signal in substantially real time.

7. A device comprising means for differentially measuring at least two MR signals.

8. The device of claim 7 further comprising means for amplifying at least two MR signals.

9. The device of claim 7 wherein at least two signals are measured simultaneously.

10. The device of claim 7 wherein the MR signals are measured after a stimulus.

11. A method for measuring a MR perturbation comprising the step of differentially measuring MR signals from at least two receivers from an object.

12. The method of claim 11 wherein at least one receiver receives MR signals from a reference location and at least one receiver receives MR signal from a test location.

13. The method of claim 12 further comprising the step of applying RF to the reference locations and the test locations.

14. The method of claim 13 wherein the RF produces free induction decay data from the reference locations and the test locations.

15. The method of claim 14 further comprising the step of converting the free induction decay to a series of phase or magnitude measurements per time period.

16. The method of claim 14 wherein the free induction decay data is analyzed in substantially real time.

17. The method of claim 14 wherein the free induction decay data is analyzed in less than 10 seconds.

18. The method of claim 11 wherein the MR signals are measured immediately after a stimulus.

19. The method of claim 18 wherein the stimulus is selected from the group consisting of a visual image, a visual sequence, an auditory sound, an, auditory sequence, a tactile sensation, an electrical stimulus to a peripheral location, an electrical stimulus to the central or peripheral nervous system, a pharmacological or other physiological stimulus, a perceptual stimuli, an instruction, and a set of instructions.

20. A programmable computer or software that performs the method of claim 19.
